(19) United States
(12) Patent Application Publication
(10) Pub. No.: US 2026/0086000 A1
(43) Pub. Date: Mar. 26, 2026

(54) FLUIDIC CIRCUIT WITH INJECTOR-SAMPLER FOR URINE ANALYZER

(71) Applicant: Withings, ISSY LES MOULINEAUX (FR)

(72) Inventors: Charlotte LÉGER, ISSY LES MOULINEAUX (FR); Antoine MERLOT, ISSY LES MOULINEAUX (FR); Aniss BENDJOUDI, ISSY LES MOULINEAUX (FR)

(21) Appl. No.: 19/340,108

(22) Filed: Sep. 25, 2025

(30) Foreign Application Priority Data
Sep. 26, 2024 (FR) FR2410306

(51) Int. Cl. G01N 1/14; E03D 9/00; G01N 33/493

(52) U.S. Cl. CPC G01N 1/14; E03D 9/00; G01N 33/493; G01N 2001/1463

(57) ABSTRACT

A urine analysis station includes a case, configured to be placed entirely within a toilet bowl and to receive a stream of urine, the case being configured to receive therein at least one analysis region adapted to receive urine for analysis, a urine reservoir, configured to receive urine from a user's urination, a fluidic circuit within the case for the circulation of urine in the station from the urine reservoir to an analysis region, an analyzer, mounted within the case, and configured to obtain data relating to the urine in the analysis region.

### CROSS-REFERENCE TO RELATED APPLICATIONS

This application claims priority to French Patent Application No. FR2410306, filed September 26, 2024, the entire content of which is incorporated herein by reference in its entirety.

### FIELD

The present invention relates to urine analysis devices and in particular to urine analysis devices comprising a case configured to be placed entirely within a toilet bowl.

### BACKGROUND

Many biological parameters can be identified in an individual's urine. For example, health problems such as urinary tract infection, diabetes or kidney failure can be detected from a urine sample. The urine sample can also reflect the quality of a diet, identify a period of fertility or pregnancy, and detect drug or tobacco use. It is therefore interesting to periodically monitor various biological parameters.

Document WO2021/175909 describes a urine analysis device. The device is housed in a toilet and collects a urine sample prior to optical analysis. The device comprises a station and a cartridge, also known as a rotatable holder, which can be removed and replaced from the station. The cartridge contains urine strips, i.e. strips coated or impregnated with a reagent that reacts with urine.

One difficulty associated with these devices is the contamination of urine by various components, in particular urine not related to the urination preceding the analysis (urine from another person or urine from a previous urination, which has a different composition and no longer enables a relevant analysis). This is known as cross-contamination.

### SUMMARY

The present description therefore discloses a urine analysis station for limiting the effects of cross-contamination.

Several solutions for limiting the effects of cross-contamination will be presented. These solutions are not mutually exclusive and can be combined.

According to one aspect, called "injector-sampler", an aspect of the description refers to a urine analysis station comprising:

- a case configured to be placed entirely inside a toilet bowl and to receive a stream of urine, the case being configured to receive within it at least one analysis region suitable for receiving urine for analysis,

- a urine reservoir, configured to receive urine from a user's urination,

- a fluidic circuit inside the case for the circulation of urine in the station from the urine reservoir to an analysis region, wherein the fluidic circuit comprises an injection end configured to inject urine into an analysis region and a sampling end configured to sample urine from the reservoir into the fluidic circuit,

- an analyzer, mounted inside the case, and configured to obtain data relating to the urine in the analysis region,

According to this "injector-sampler" aspect, the injection end is used as the sampling end. In this way, the injection tip is rinsed with the user's urine prior to injection.

In an embodiment, the injection tip is movable between an injection position, wherein the injection tip injects urine into the region of analysis, and a sampling position, wherein the injection tip samples urine from the reservoir.

In an embodiment, the reservoir and the at least one analysis region are arranged in close proximity in the station, for example in alignment with a translation axis of the injection end, which is therefore mobile. By “close proximity” is meant that the reservoir and the analysis region are positioned such that the injection end can move directly between the two locations along a substantially linear path without requiring intermediate redirection, typically separated by a distance less than 30 millimeters, and in some embodiments less than 15 millimeters. This spatial arrangement minimizes dead volume in the fluidic circuit, reduces travel time of the injection end, and improves reliability of urine transfer. In alternative embodiments, “close proximity” may also be defined functionally as an arrangement in which the injection end can reach both the reservoir and the analysis region using a single actuator stroke or a single degree of translational movement.

In an embodiment, the injection end is a syringe.

In an embodiment, the station comprises a pump configured to circulate or convey urine in the fluidic circuit in one direction during a step of urine collection from the reservoir by the sampling end and in the opposite direction during a step of urine injection into the analysis region by the injection end. In particular, the pump can be configured to reverse its direction of operation.

In an embodiment, the fluidic circuit is linear, i.e. without any bypasses or branches.

In an embodiment, the fluidic circuit forms a loop between the injection end and the sampling end.

In an embodiment, the station is configured to sacrifice the first volumes of urine taken from the reservoir by the sampling end, so that the urine station does not inject the first volumes of urine taken into the analysis region.

In an embodiment, the fluidic circuit comprises a section for changing the direction of movement of the urine, wherein the urine changes direction of flow, i.e. wherein a leading front of a volume of urine becomes the trailing front of this volume of urine.

In an embodiment, the logic of the fluidic circuit is "last in - first out", LIFO, so that the last volumes of urine collected by the sampling end in the reservoir are the first volumes of urine injected into the analysis region by the injection end.

In an embodiment, the station comprises a space positioned inside the case, configured to at least partially receive a cartridge comprising the at least one analysis region.

In an embodiment, the reservoir is accessible from the sampling end via a septum configured to be traversed by the sampling end.

This aspect also relates to a device comprising a station as previously described and a cartridge removable from the station, wherein the at least one analysis region is mounted on the cartridge.

This aspect also relates to a method of urine analysis using a station or device as previously described, the method comprising: - a step of sampling urine from the reservoir through the sampling end, - a step of injecting urine into the analysis region via the injection end.

In an embodiment, the method comprises, between the sampling step and the injecting step, a pre-charging step, during which sampled urine is returned to the reservoir via the sampling end.

According to a so-called "sacrificial" aspect, the description relates to a urine analysis station comprising:

- a case configured to be placed entirely within a toilet bowl and to receive a stream of urine, the case being configured to receive within it at least one analysis region suitable for receiving urine for analysis,

- a urine reservoir, configured to receive urine from a user's urination,

- a fluidic circuit inside the case for the circulation or conveyance of urine in the station from the urine reservoir to an analysis region, wherein the fluidic circuit comprises an injection end configured to inject urine into an analysis region and a sampling end configured to sample urine from the reservoir into the fluidic circuit,

- an analyzer, mounted inside the case, and configured to obtain data relating to the urine in the analysis region.

According to this aspect, the station is configured to sacrifice the first volumes of urine taken from the reservoir by the sampling end, so that the urine analysis station does not inject the first volumes of urine taken into the analysis region.

In an embodiment, the station further comprises a drain end, the station being configured so that: - in a drain position, the injection end discharges urine to the drain end, then, - in an injection position, the injection end injects urine into the analysis region.

In an embodiment, the fluidic circuit is linear, with the drain end opposite the sampling end.

In an embodiment, the fluidic circuit is configured to circulate urine in a unidirectional direction of movement, from the sampling end to the injection end.

In an embodiment, the injection end is used as a sampling end, wherein urine enters the fluidic circuit, so that the injection end is flushed with the user's urine prior to injection.

In an embodiment, the fluidic circuit is arranged so that urine flows through the injection end in one direction during collection and in the other direction during injection.

In an embodiment, the sacrificial volumes are drained from the fluidic circuit after the injecting step.

In an embodiment, the first volumes injected are the last volumes of urine collected or intermediate volumes of urine collected.

In an embodiment, the fluidic circuit comprises a direction-reversal section for changing the direction of movement of the urine, wherein the urine changes direction of circulation or conveyance, and wherein a leading front of a volume of urine becomes the trailing front of this volume of urine. For example, the direction-reversal section is configured such that, between a urine-sampling operation and a urine-delivery operation, a collected bolus of urine reverses flow direction in the direction-reversal section so that a leading front of the bolus during sampling becomes a trailing front during delivery. As used herein, the term ‘bolus of urine’ refers to a discrete volume of urine that is at least partially bounded by fluidic interfaces, such as air gaps, valve closures, or flow reversals, such that the volume is transported through the circuit as a unit.

In an embodiment, the station comprises a space positioned inside the case, configured to at least partially receive a cartridge comprising the at least one analysis region.

This aspect also relates to a device comprising a station as previously described and a cartridge removable from the station, wherein the at least one analysis region is mounted on the cartridge.

This aspect also relates to a method of urine analysis using a station or device as previously described, the method comprising:

- a step of sampling urine from the reservoir through the sampling end, the sampling comprising first sampled volumes,

- a step of injecting urine into the analysis region through the injection end, the injection not including the first sampled volumes (i.e. including the last sampled urine volumes or intermediate sampled urine volumes).

In an embodiment, the method comprises, between the sampling step and the injecting step, a sacrificing step, during which the first volumes sampled are discharged into a drain end, notably via the injection end.

In an embodiment, the method comprises, after the injecting step, a draining step during which the first sampled volumes are sent to the draining end.

According to a so-called "two-way" aspect, the description refers to a urine analysis station comprising:

- a case configured to be placed entirely within a toilet bowl and to receive a stream of urine, the case being configured to receive within it at least one analysis region suitable for receiving urine for analysis,

- a fluidic circuit, inside the case, for the circulation or conveyance of urine in the station to an analysis region,

- an analyzer, mounted inside the case, and configured to obtain data relating to the urine in the analysis region.

According to this aspect, the fluidic circuit comprises a section for changing the direction of movement of urine, within which urine changes direction of circulation or conveyance, within which a leading front of a volume of urine becomes a trailing front of this volume of urine.

In this respect, the station may comprise a pump configured to cause the direction of fluid flow in the urine flow direction change section to change. This can be achieved by reversing the direction of pump operation.

In an embodiment, the station comprises a urine reservoir, configured to receive urine from a user's urination, and the fluidic circuit is for the circulation or conveyance of urine in the station from the urine reservoir to an analysis region.

In an embodiment, the fluidic circuit comprises an injection end configured to inject urine into an analysis region and a sampling end configured to collect urine from the reservoir and feed it into the fluidic circuit.

In an embodiment, the logic of the fluidic circuit is "last in - first out", LIFO, so that the last volumes of urine taken from the reservoir by the sampling end are the first volumes of urine injected into the analysis region by the injection end, so that the urine last in is the cleanest and the one injected.

In an embodiment, the injection end is used as a sampling end.

In an embodiment, the section for changing the direction of movement of the urine comprises the injection end, so that the injection end is rinsed with the user's urine prior to injection.

In an embodiment, the station comprises two fluid presence sensors disposed at two locations along the fluidic circuit, wherein the urine displacement change section comprises the portion of the fluidic circuit between the two fluid presence sensors.

In an embodiment, the station is configured to sacrifice the first urine volumes collected from the reservoir by the sampling end, so that the station does not inject the collected first urine volumes into the analysis region.

In an embodiment, the reservoir and the at least one analysis region are arranged in close proximity in the station. By “close proximity” is meant that the reservoir and the analysis region are located sufficiently near one another such that the injection end can access both without complex routing or multi-axis repositioning. In practical terms, this may correspond to a physical spacing of less than about 30 millimeters between the reservoir and the analysis region, and in certain embodiments less than about 15 millimeters. Functionally, “close proximity” encompasses arrangements in which the injection end can move between the reservoir and the analysis region along a substantially linear translation axis, with minimal dead volume in the fluidic circuit. This spatial configuration ensures efficient transfer of urine, reduces mechanical complexity, and improves accuracy and reliability of analysis.

In an embodiment, the station comprises a space positioned inside the case, configured to at least partially receive a cartridge comprising the at least one analysis region.

This aspect also relates to a device comprising a station as previously described and a cartridge removable from the station, wherein the at least one analysis region is mounted on the cartridge.

This aspect also relates to a method of urine analysis using a station or device as previously described, the method comprising:

- a step of sampling urine from the reservoir through the sampling end,

- a step of injecting urine into the analysis region through the injection end, wherein between the sampling step and the injecting step, the urine changes direction of flow in the urine direction of travel change section.

In an embodiment, the station comprises a pump, and the direction of pump operation is reversed between the sampling step and the injecting step.

In an embodiment, the method comprises, between the sampling step and the injecting step, a pre-charge step, during which sampled urine is returned to the reservoir via the sampling end.

In particular, all these solutions can be implemented with the features shown below, unless the description specifies otherwise.

The fluidic circuit has characteristic dimensions such that the capillarity forces are greater than the forces of gravity. Consequently, in the absence of any external force other than gravity, urine does not circulate or convey in the fluidic circuit.

In an embodiment, the station comprises a space positioned inside the case, configured to at least partially receive a cartridge comprising the at least one analysis region. The cartridge may comprise a plurality of analysis regions, each configured to be selectively positioned facing the injection end.

In an embodiment, the space comprises an annular shape and the cartridge is rotatable in the station.

The description also relates to a device comprising a station as described herein, and a cartridge, wherein the at least one analysis region is mounted on a removable cartridge of the station.

The station may comprise a collection port positioned on the case and the sampling end samples from the reservoir via the collection port.

The reservoir may be formed in part by the case. In an embodiment, the reservoir is on the outside of the case, in order to receive urine flowing onto the case. In this respect, the reservoir is a buffer reservoir (urine passes through it and is collected by the sampling end) or a storage reservoir where urine accumulates.

In an embodiment, the reservoir is accessible from the sampling end via a septum configured to be traversed by the sampling end.

The station typically comprises a pump configured to circulate or convey fluid in the fluidic circuit, for example to circulate or convey it in one direction or the other. The pump may be a peristaltic pump, which keeps the urine in the piping.

In an embodiment, the sampling end comprises a needle. The needle may be beveled, in particular to pierce the septum and to pierce the cartridge chamber.

In an embodiment, the injection end is movable inside the case, for example in translation, by means of a motor.

In an embodiment, the reservoir and the at least one analysis region are arranged in close proximity (when the analysis region is positioned for the injecting step), for example in alignment with the translation axis of the injection end. Alternatively, the drain end and the at least one analysis region are arranged in close proximity (when the analysis region is positioned for the injecting step), for example in alignment with the translation axis of the injection end.

In an embodiment, the drain end is fluidly opposite the injection end.

In an embodiment, the fluidic circuit is linear, i.e. without branches or junctions.

In an embodiment, the injection end is movable between an injection position, wherein the injection end injects urine into the analysis region, and a sampling position, wherein the injection end samples urine from the reservoir. Alternatively, the injection end may be movable between an injection position and a drain position, during which the injection end discharges urine to a drain end.

In an embodiment, the station is configured to implement a pre-charge step, between the sampling step and the injecting step, during which the last volumes of urine collected are reinjected into the reservoir. The pre-charge step can be implemented in response to a determination that a reference section between the two fluid presence sensors indicated that the reference section contained only urine.

The station comprises control circuitry, with a processor and memory capable of storing a program that the processor can execute. In particular, the control circuitry is configured to control the position of the injection end (between the different positions) to control the pump and able to control the position of the at least one analysis region in the case.

The analyzer typically comprises an optical analyzer, for example a CCD sensor or a camera.

In an embodiment, the station comprises a space positioned inside the case, configured to at least partially receive a cartridge comprising the at least one analysis region. The space may be annular in shape. The cartridge is typically rotatable.

In an embodiment, there is provided a urine analysis station comprising: a case configured to be disposed entirely within a bowl of a toilet and to receive a stream of urine; a fluidic circuit disposed within the case and configured to convey urine to at least one analysis region; and an analyzer within the case and configured to obtain data from urine at the analysis region; wherein the fluidic circuit includes a direction-reversal section configured such that, between a urine-sampling operation and a urine-delivery operation, a collected bolus of urine reverses flow direction in the section so that a leading front of the bolus during sampling becomes a trailing front during delivery.

In an embodiment, there is provided urine analysis station comprising: a case configured to be disposed entirely within a bowl of a toilet and to receive a stream of urine; a fluidic circuit within the case and configured to convey urine from a reservoir to at least one analysis region; an injection end within the case positionable to deliver urine to the analysis region; an analyzer within the case and configured to obtain data from urine at the analysis region; and a pump operatively coupled to the fluidic circuit and controllable to change a flow direction of urine in a direction-reversal section of the circuit, whereby, after sampling, the flow direction is reversed so that last-collected urine is delivered first to the analysis region.

The description may also relate to a urine analysis device comprising a station as previously described and a cartridge as previously described.

### BRIEF DESCRIPTION OF THE DRAWINGS

Further features, details and benefits will become apparent from the detailed description below, and from an analysis of the appended drawings, in which:

- FIG. 1 shows a simplified schematic representation of a urine analysis device installed in a toilet bowl,

- FIG. 2 shows an exploded view of the urine analysis device, wherein the station and cartridge are visible,

- FIG. 3 shows a detailed view of a cartridge in an embodiment,

- FIG. 4 shows a cross-sectional view of a cartridge and station according to one embodiment, at the location of an optical analyzer of the station,

- FIG. 5 shows two embodiments of a cross-sectional view of the device (station and cartridge) at the injection end,

- FIG. 6 shows schematic views of the fluidic circuit of a measuring station conforming to a so-called "sacrificial" embodiment, new paragraph...

- FIG. 7 shows a more realistic view of a fluidic circuit similar to that of FIG. 6,

- FIG. 8 shows schematic views of the fluidic circuit of a measuring station conforming to a so-called "injector-sampler" embodiment,

- FIG. 9 shows schematic views of the fluidic circuit of a measuring station conforming to "sacrificial" and "injector-sampler " embodiments,

- FIG. 10 shows schematic views of the fluidic circuit of a measuring station conforming to a "two-way" embodiment (and suitable for the "sacrificial" embodiment),

- FIG. 11 shows schematic views of the fluidic circuit of a measuring station conforming to the "injector-sampler ", "two-way" and "sacrificial" embodiments,

- FIG. 12 shows a more realistic view of a fluidic circuit similar to that shown in FIG. 11,

- FIG. 13 shows a simplified view of the fluidic circuit of FIG. 12, in a sampling stage,

- FIG. 14 shows a simplified view of the fluidic circuit of FIG. 12, in an injection stage,

- FIG. 15 shows a simplified view of the fluidic circuit of FIG. 12, in a draining stage,

- FIG. 16 shows a schematic architecture of a measuring station and its ecosystem.

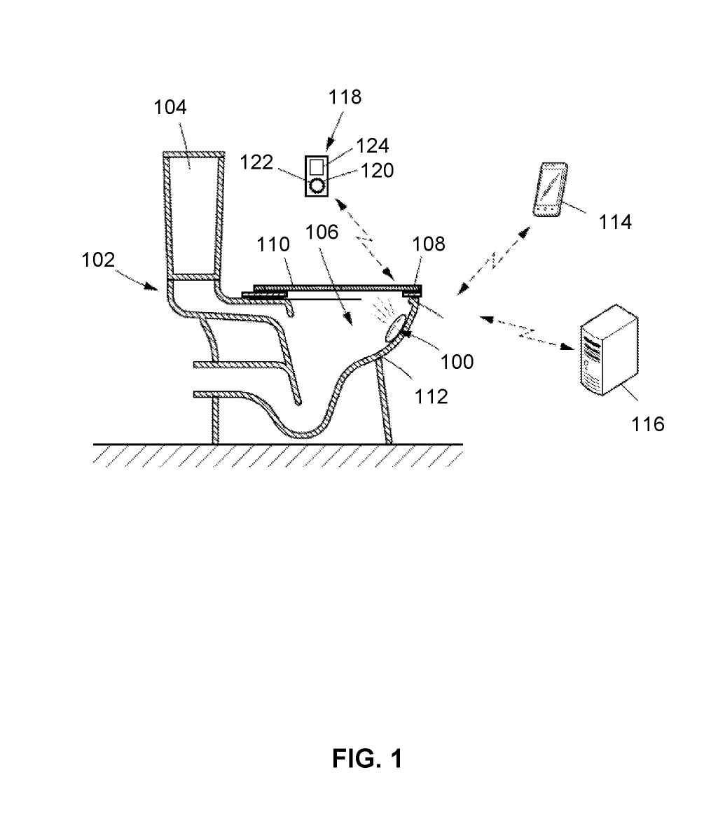

FIG. 1 shows a simplified schematic representation of a urine analysis device installed in a toilet bowl,

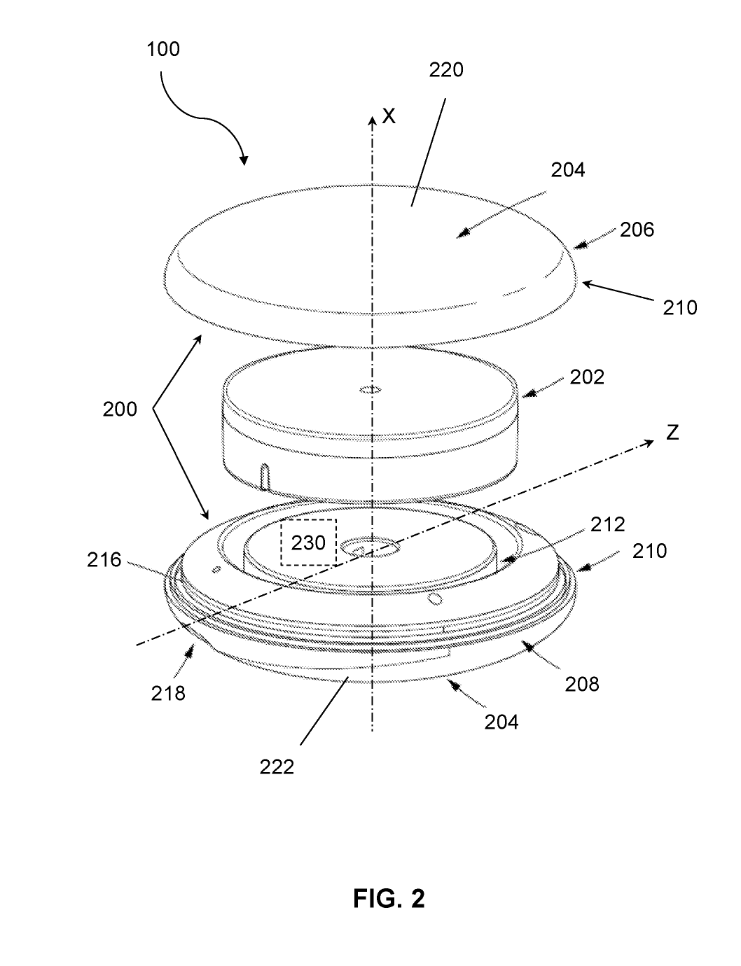

FIG. 2 shows an exploded view of the urine analysis device, wherein the station and cartridge are visible,

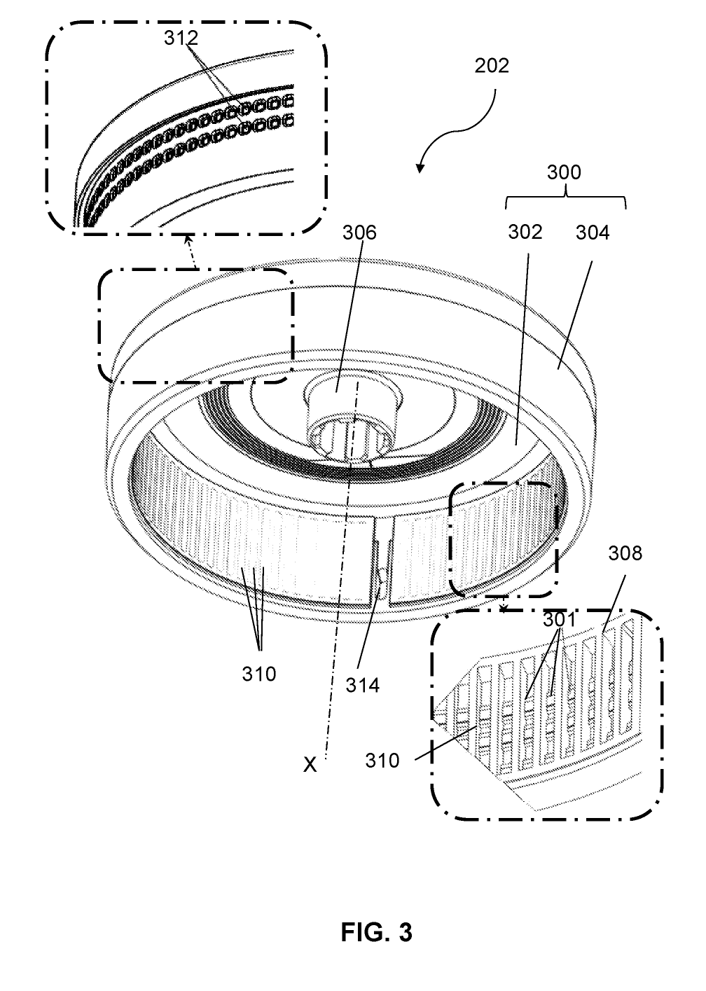

FIG. 3 shows a detailed view of a cartridge in an embodiment,

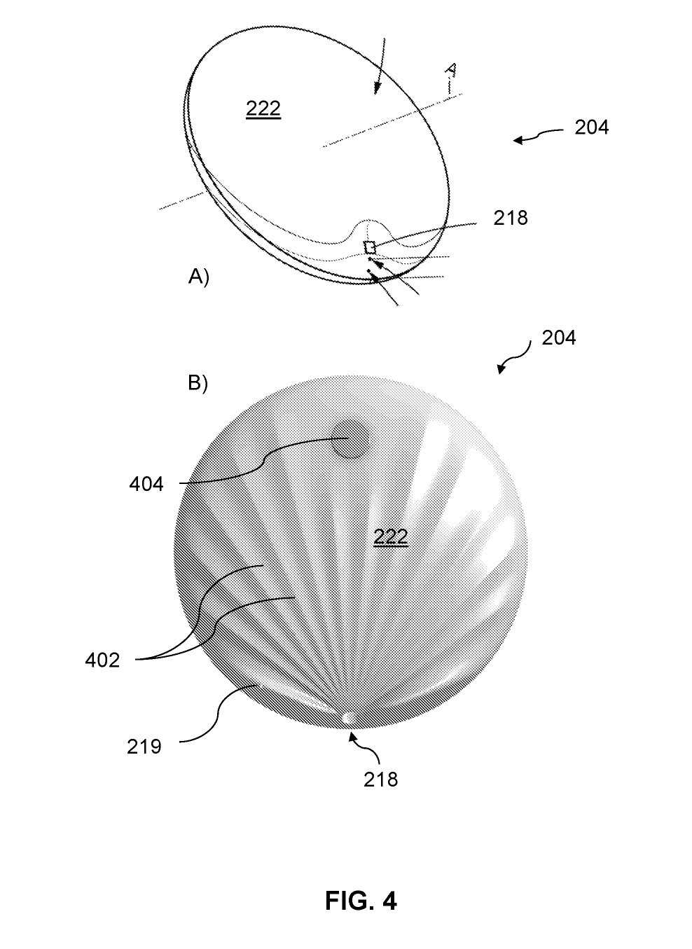

FIG. 4 shows a cross-sectional view of a cartridge and station according to one embodiment, at the location of an optical analyzer of the station,

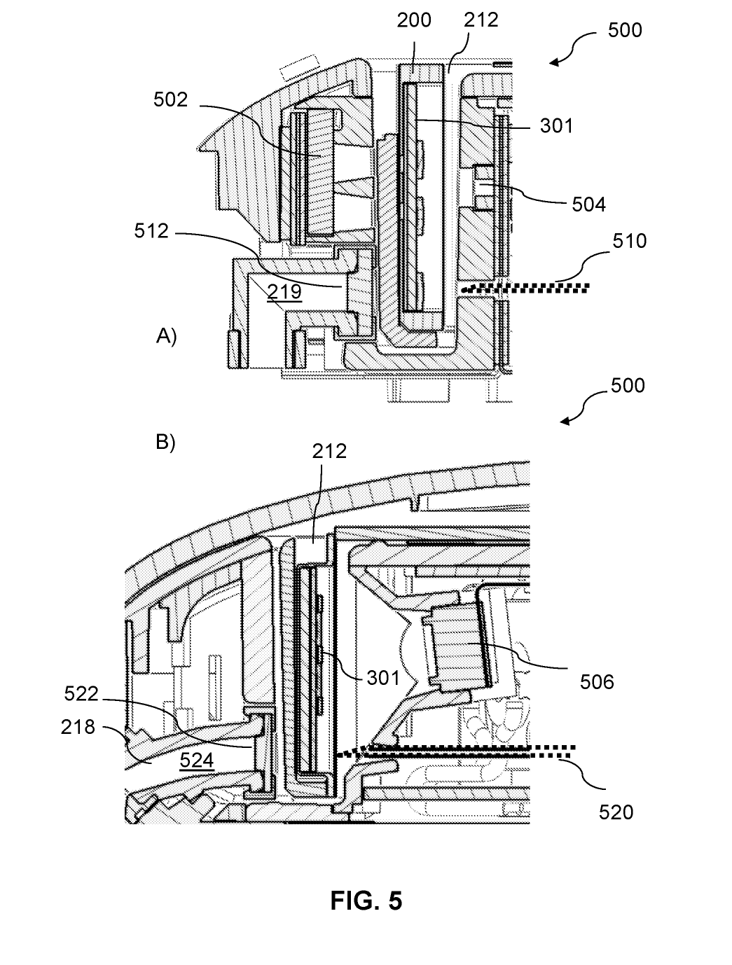

FIG. 5 shows two embodiments of a cross-sectional view of the device (station and cartridge) at the injection end,

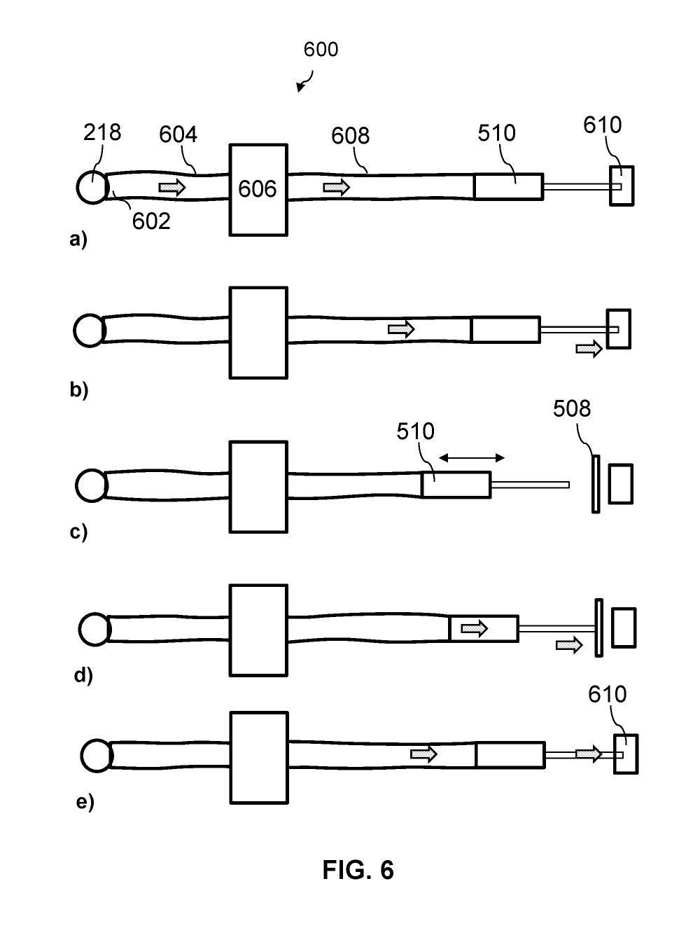

FIG. 6 shows schematic views of the fluidic circuit of a measuring station conforming to a so-called "sacrificial" embodiment,

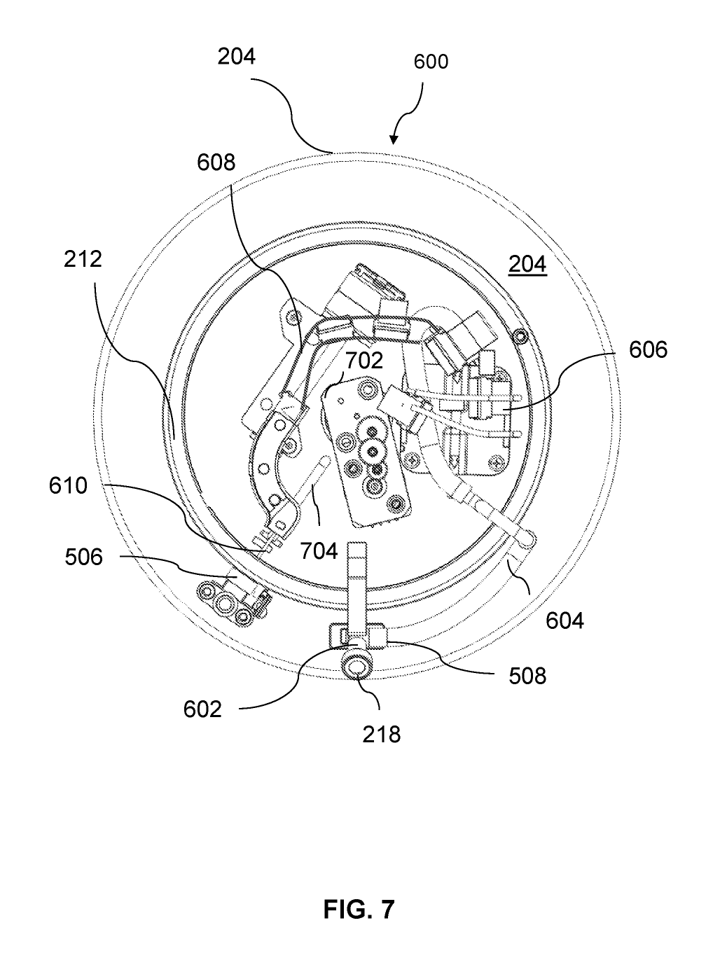

FIG. 7 shows a more realistic view of a fluidic circuit similar to that of FIG. 6,

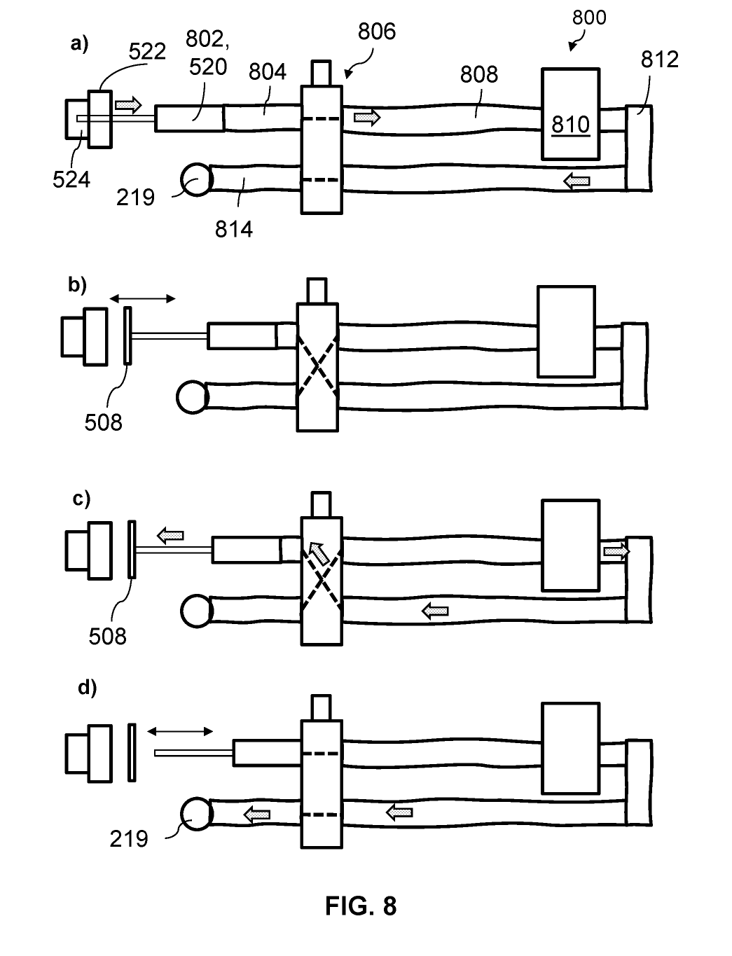

FIG. 8 shows schematic views of the fluidic circuit of a measuring station conforming to a so-called "injector-sampler" embodiment,

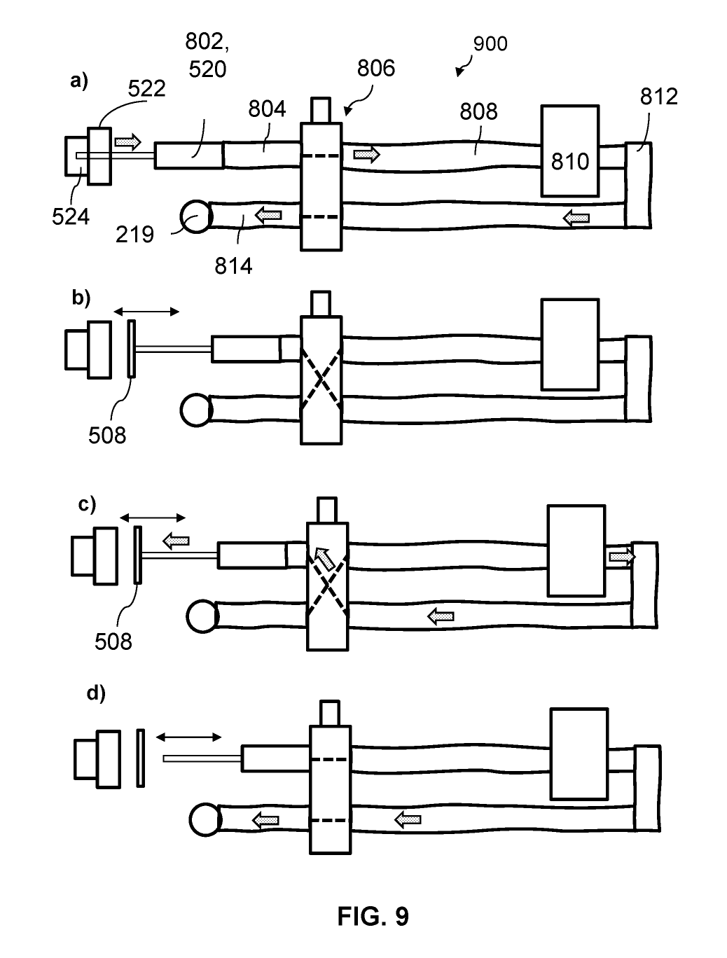

FIG. 9 shows schematic views of the fluidic circuit of a measuring station conforming to "sacrificial" and "injector-sampler " embodiments,

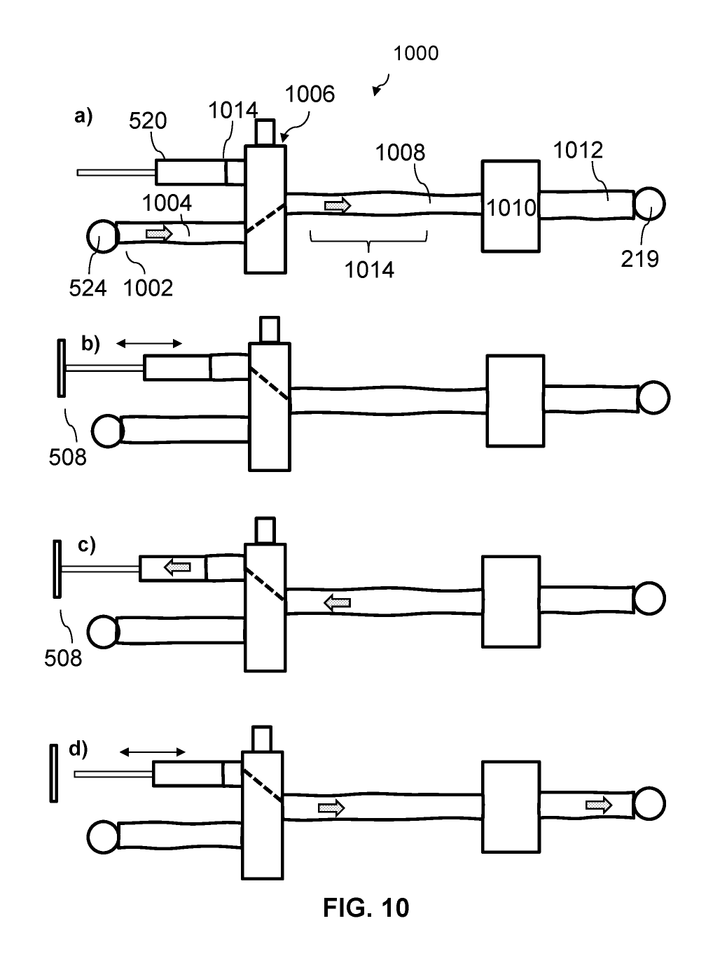

FIG. 10 shows schematic views of the fluidic circuit of a measuring station conforming to a "two-way" embodiment (and suitable for the "sacrificial" embodiment),

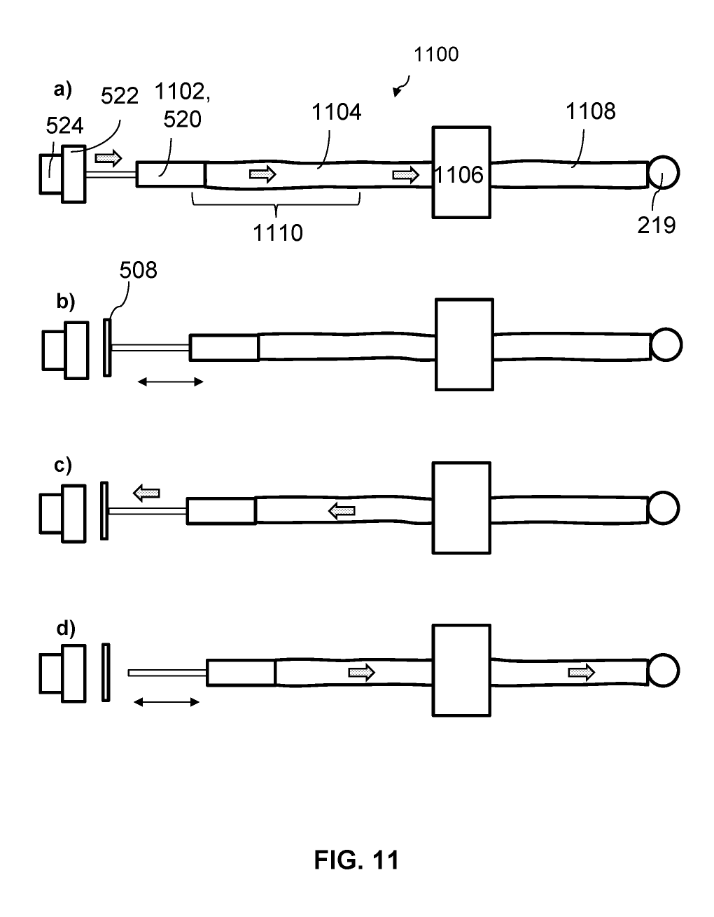

FIG. 11 shows schematic views of the fluidic circuit of a measuring station conforming to the "injector-sampler ", "two-way" and "sacrificial" embodiments,

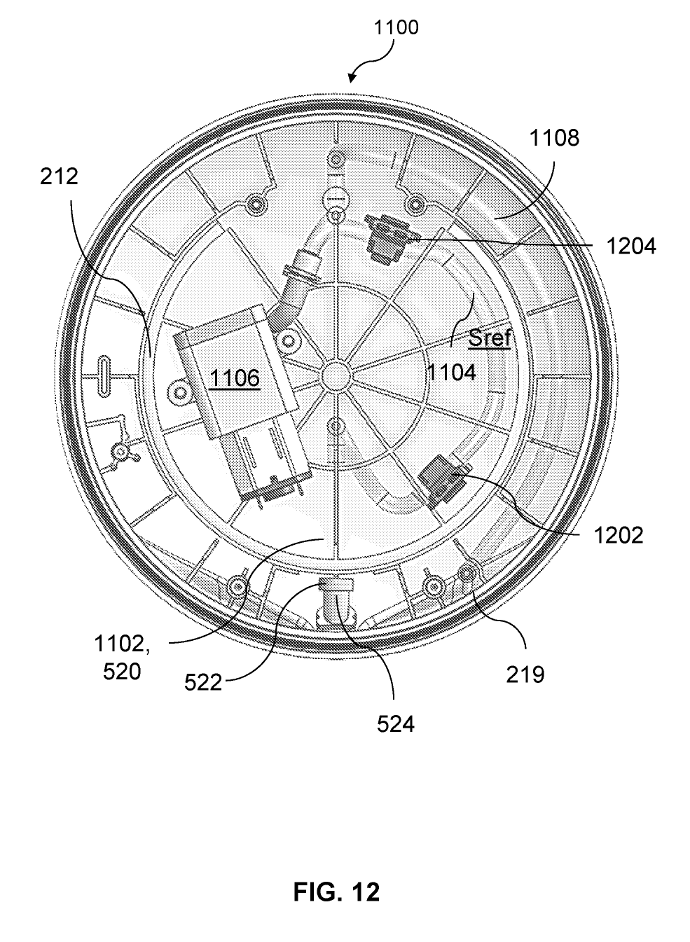

FIG. 12 shows a more realistic view of a fluidic circuit similar to that shown in FIG. 11,

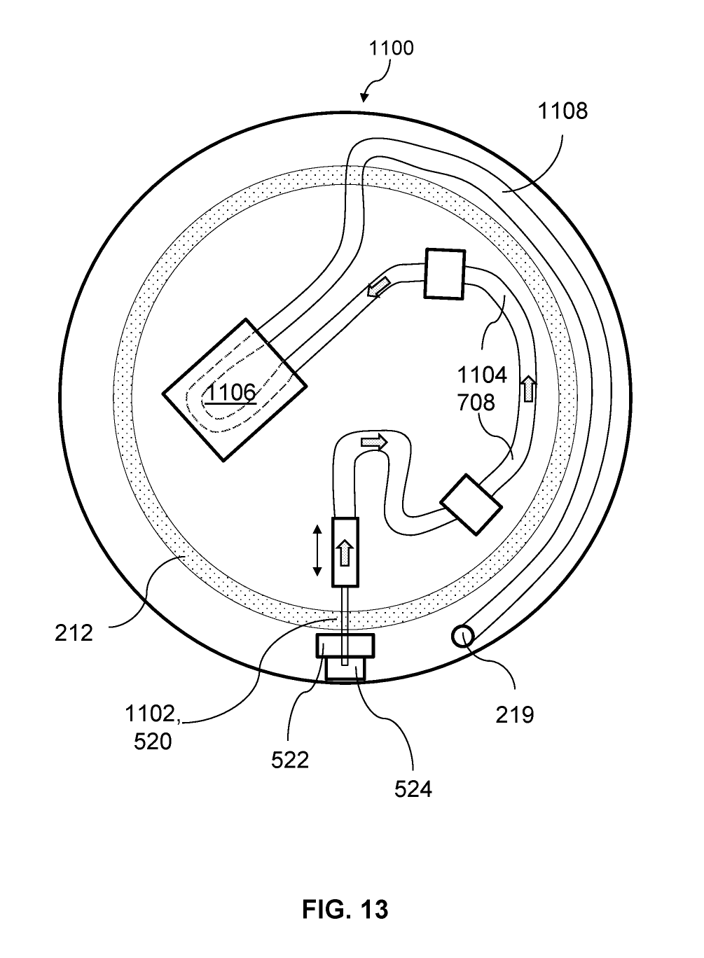

FIG. 13 shows a simplified view of the fluidic circuit of FIG. 12, in a sampling stage,

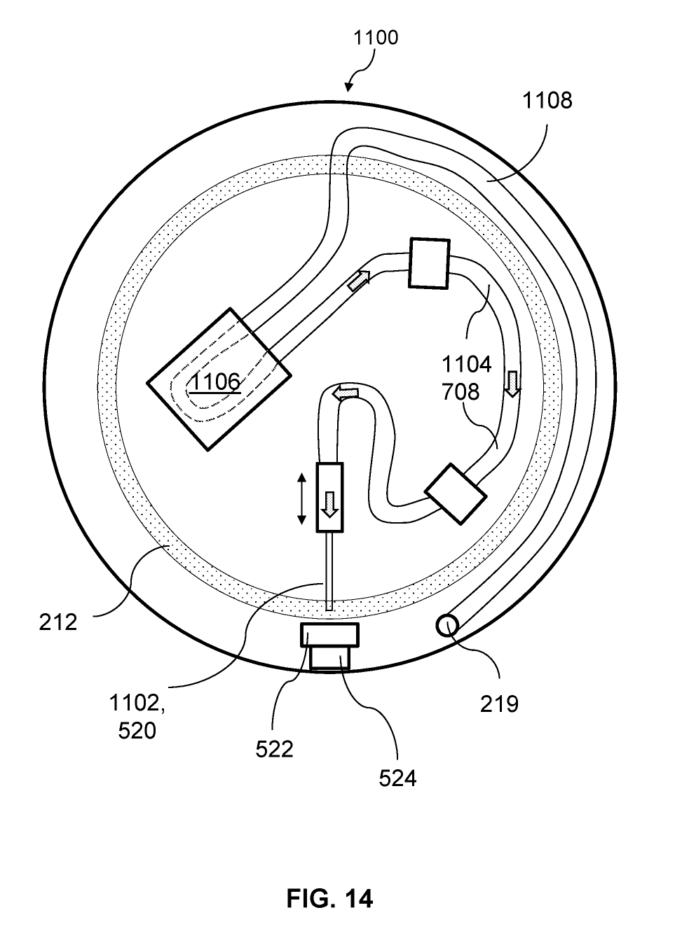

FIG. 14 shows a simplified view of the fluidic circuit of FIG. 12, in an injection stage,

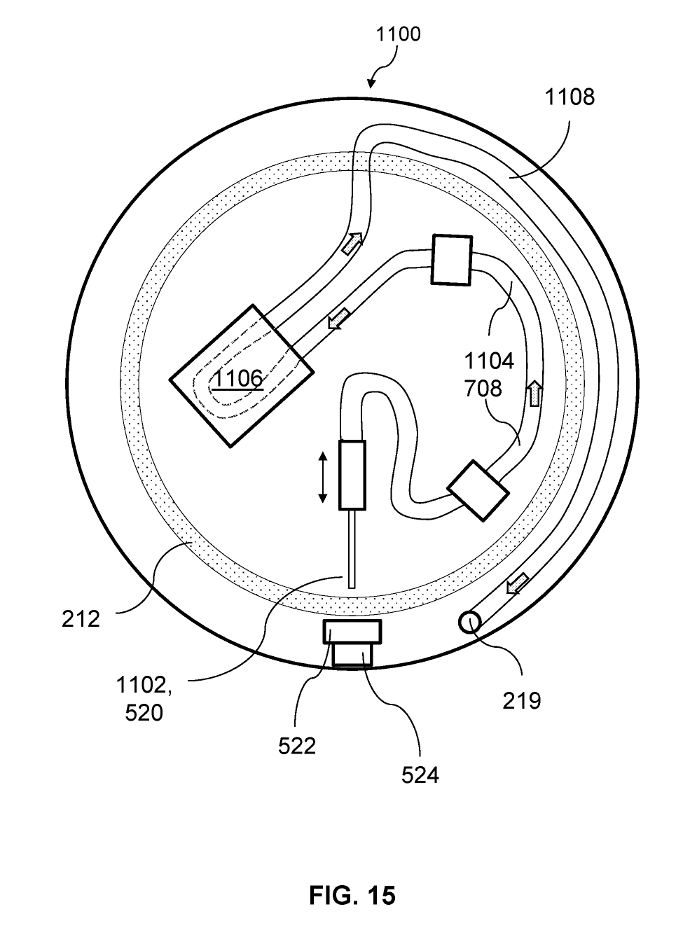

FIG. 15 shows a simplified view of the fluidic circuit of FIG. 12, in a draining stage,

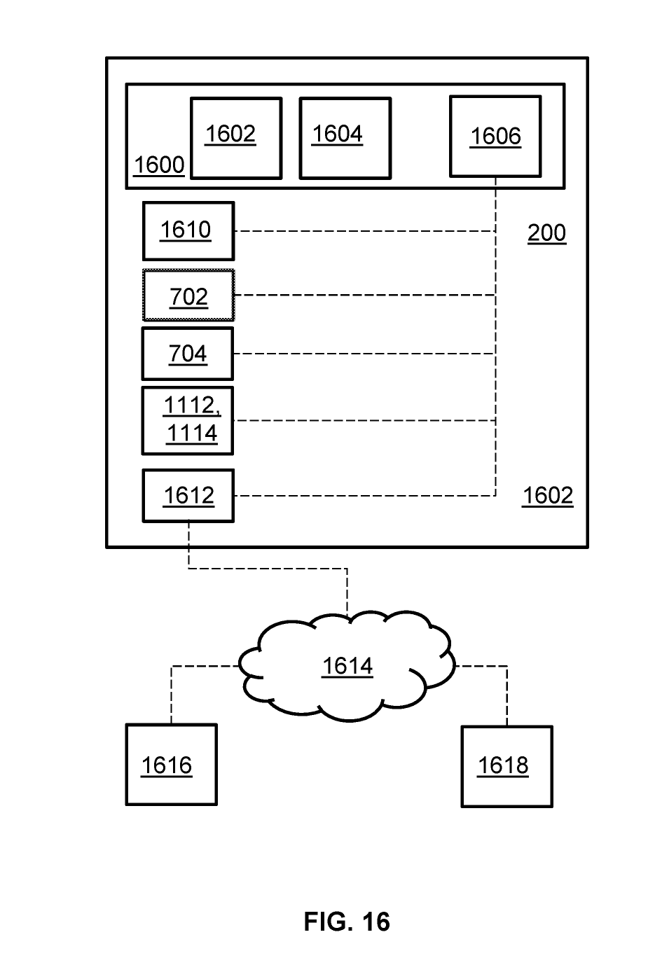

FIG. 16 shows a schematic architecture of a measuring station and its ecosystem.

### DETAILED DESCRIPTION

The present description presents different fluidic circuit architectures of a urine analysis device comprising a case sized to be disposed on a wall of a toilet bowl. The following documents describe an example of such an analysis device: WO2021175909 and WO2021175944, WO2023036805, WO2023036806, WO2023036808, WO2023036809. These will hereafter be referred to as WO documents.

The function of the fluidic circuit is to convey urine collected by the case to an analysis region, which typically comprises a reagent, positioned inside the case, and in particular to a removable cartridge received in the case.

Several embodiments and variants of fluidic circuits will be presented in relation to a device conforming to the aforementioned WO documents by way of example.

### Orientation

In the following, the notions of "top" and "bottom", "upper" and "lower", etc. are defined in relation to a Z direction, as defined in FIG. 2. The top along the Z direction is defined in a normal use position of the urine analysis device fixed in the toilet bowl.

### General shape of the case

FIG. 1 schematically illustrates a urine analysis device 100 (hereinafter referred to as "device 100") installed in toilet 102. The toilet 102 typically comprises a water tank 104, a bowl 106, a seat 108 and a seat cover 110. The analysis device 100 is configured to be placed entirely within the toilet bowl. By "in the bowl" is meant "placed in the interior volume defined by the bowl". The analysis device 100 is removably arranged in the toilet 102. For example, the analysis device 100 can be easily removed from the toilet to replace a cartridge, and then replaced in the toilet 102. The analysis device 100 is placed on a wall 112 of the toilet bowl 106. The analysis device 100 is positioned so that it is generally under a user's urine stream, so that when a user urinates (generally in a seated position), the urine comes into contact with the analysis device 100. The analysis device 100 can communicate remotely with a remote entity, such as smartphone 114 or server 116.

As further illustrated in FIG. 2, the urine analysis device 100 may comprise a urine analysis station 200 (hereinafter also referred to as "station 200") and a cartridge 202, removably mounted on station 200. The cartridge 202 comprises reagent capable of reacting with urine (referred to as "urine reagent"). In an embodiment without the cartridge 202, the analysis device 100 and the analysis station 200 are one and the same.

Alternatively, analysis station 200 comprises urine reagent directly, without removable parts. Alternatively, station 200 can be refilled by pouring in reagent in liquid form.

Alternatively, urine analysis can be performed without reagent. One example is optical analysis, such as spectroscopy.

Station 200 may comprise a case 204 which may have two shells, in particular a front shell 206 and a rear shell 208. The front shell 206 and the rear shell 208 can cooperate with each other via a fastening mechanism 216, in a plane normal to the X axis. The front shell 206 and rear shell 208 can be reversibly assembled, for example by screwing or clipping. In one variant, the front shell 206 and rear shell 208 can be permanently joined, for example by gluing, clipping, magnetizing or ultrasonic welding. Other fastening methods can be used to join the two shells.

The case 204 is watertight. Only a collection port and a drain port allow urine to pass between the inside and the outside. These ports will be described in greater detail later.

As can be seen from the figures, the case 204 may have the overall external shape of a circular roller. In other words, the case 204 has a spheroidal shape. The X axis is the center line of the case. Beneficially, the front shell 206 can be substantially rotationally symmetrical, giving the device an aerodynamic appearance once installed. The case 204 serves as a urine collector.

The case 204 comprises a front face 220 for receiving a stream of urine directly from a user urinating on the toilet, and a rear face 222 opposite the front face 220. As illustrated in FIG. 2, the front face 220 can be arranged on the front shell 206 and the rear face 222 can be arranged on the rear shell 208. The front face 220 faces the inside of the bowl 106. The front face 220 is therefore intended to receive urine when the user urinates while sitting on the toilet 102. As shown in FIGS. 5 and 6, the rear face 222 faces the inner wall 112 of the bowl 106. For the rest of the description, an object facing the bowl wall is taken to mean an object facing the bowl wall closest to the object in question, and not the facing bowl wall on the other side of the inner bowl volume.

The front face 220 and rear face 222 each have a curved edge 210. The respective curved edges 210 of the front face and rear face meet at an equatorial junction zone. Thus, the outer surface of case 204, consisting of front face 220 and rear face 222, is defined by curved lines and forms a generally convex object.

The outer surface of case 204 can also be white or light-colored. The color of the outer surface can be similar to that of the toilet, which enhances the discreetness of the device.

In an embodiment, the case 204 can have a diameter, measured in the direction orthogonal to the X axis, of between 50 mm and 150 mm. In an embodiment, the case 204 can have a thickness, measured in the direction of the X axis, of between 15 mm and 50 mm. In this way, the case 204 is compact enough to be housed entirely in the toilet bowl. The urine analysis device 100 is unobtrusive. In addition, the case 204 is large enough to systematically come into contact with the urine received in the toilet bowl. The user can then urinate in the toilet without worrying about the urine analysis device, or alternatively aim summarily.

According to another aspect, in an embodiment, the case 204 has a general form factor such that the ratio between thickness and diameter is between 0.2 and 0.5, for example between 0.3 and 0.4. Such proportions are reminiscent of a natural pebble and give the device a soothing appearance. The spheroidal ‘pebble’ shape minimizes splash-back and offers low resistance to water flow, encouraging complete and uniform flushing.

Case 204 may be made of a hydrophilic material. For example, the material of case 204 may be ceramic, polyamide (PA), silicone or a hydrophilic polymer. The outer surface of case 204 can also be treated with a hydrophilic surface treatment, for example AcuWet® from Aculon, a hydrophilic polymer, or Pebax® from Arkema.

The station 200 comprises a collection port 218, located for example on the rear shell 208. As will be explained in more detail below, the collection port 218 is configured to collect urine dripping onto the surface of the case 204. Station 200 also comprises a drain, whose drain port 219 is visible in particular in FIGS. 4B) and 5A), configured to drain liquid out of device 100. The rear-facing collection port and spacer arrangement prevents direct exposure to user urine streams and flush surges, reducing fouling risk and ensuring sensor longevity. This also avoids turbulent flow disruption during sample intake, improving test accuracy.

### Cartridge space

Station 200 typically comprises an annular compartment 212, located inside case 204, arranged around an axis of rotation X. The annular compartment 212 is configured to at least partially receive the cartridge 202 rotatably mounted around the axis of rotation X (once in position in the annular compartment 212). The cartridge comprises a plurality of analysis regions.

In the embodiment shown in the figures, cartridge 202 comprises urinary reagent, in particular by means of a plurality of test carriers which each comprise at least one urinary reagent, for example a dry reagent. In the illustrated example, the plurality of test carriers is arranged along a circle or circular arc around the axis of rotation X and form the plurality of analysis regions. In an embodiment, the test supports are test strips. The test carriers can be enclosed, for example individually, in a sealed chamber.

Alternatively, cartridge 202 comprises chambers serving as independent volumes for receiving urine to undergo optical analysis of the urine directly. The chambers then form the plurality of analysis regions.

The annular compartment 212 typically extends 360° and forms a groove configured to at least partially receive the cartridge 202.

EP4338839 describes a method for obtaining sealed chambers in a cartridge.

In an embodiment, all analysis regions are at the same location in the case to receive urine and/or be analyzed (as illustrated, with rotation of the cartridge). Alternatively (not shown), the analysis regions can be at different locations inside the case for receiving urine and/or being analyzed.

### Test set

A test set is arranged inside case 204 and configured to perform an analysis on urine collected through the collection port.

In particular, the test assembly comprises a fluidic circuit, configured in particular to convey urine from a urine reservoir to the at least one analysis region. The urine reservoir is typically formed by the case. The urine then enters the fluidic circuit through the collection port. In an embodiment that will be described later, the collection orifice is blocked by a septum that can be penetrated by a needle. The fluidic circuit comprises an injection end (also known as an injector) and the collection orifice. Station 200 further comprises a pump (or pump system) to set the fluid in motion in the fluidic circuit. The pump can be a peristaltic pump, which keeps the urine in the piping (more convenient for cleaning and to avoid cross-contamination).

The test set further comprises an analyzer 230. The pump draws urine through the collection port 218, then the injection end injects the urine into an analysis region, for example onto urine reagent or into a chamber. The injection end is thus configured to deposit urine in the analysis region. In an embodiment, the analysis region can be positioned, for example by rotating the cartridge, opposite the analyzer 230 to perform the analysis. Finally, the analyzer measures certain property values (for example, physical/chemical properties, such as color) of the reagent after it has come into contact with the urine, or of the urine directly. In an embodiment, the analyzer is an optical analyzer (e.g. a camera) configured to analyze the optical properties of the reagent. In other embodiments, the optical analyzer can be configured to perform spectroscopy of the urine. As a result, the analyzer is configured to obtain data relating to the urine in the analysis region, either data obtained directly from the urine or data obtained indirectly from the urine (via the reagent).

The injection end and the cartridge can move relative to each other, in particular to be able to select an analysis region (e.g. a reagent) which is to receive urine from the injection end, and for the injection end to be able to open (e.g. pierce) the chamber, for example using a needle or needle-like device. The previously cited WO documents detail the movement of the injection end (referred to as an injector or syringe in the WO documents).

Station 200 comprises control circuitry 1600, illustrated in FIG. 16, capable of controlling the various components of device 100, such as the position of the injection end or the activation of the pump or, where applicable, valve.

### Cartridge

FIG. 3 shows an exploded view of a cartridge 202. The cartridge 202 comprises at least one analysis region, for example at least one test support 301, in particular several reaction zones (in particular several test supports 301) configured to receive urine from the injector. In an embodiment, each test carrier 301 contains a urine reagent that reacts in a specific way on contact with urine.

The cartridge 202 comprises a rotatable holder 300, configured to be driven in rotation by the station 200, for example by a motor (reference 702 in FIG. 7). During normal use of the cartridge 202 and the device 100, the reaction zones (i.e. the test holders 301) remain attached to the rotatable holder and do not move relative to it.

In an embodiment, the rotatable holder 300 has a straight circular cylinder shape of at least 80% of a hollow cylinder shape extending annularly around an axis which is, when the cartridge 202 is mounted in the station 200, the axis of rotation X. Each test support 301 may be a test strip. The rotatable holder 300 may comprise an annular part 302 and a cylindrical portion 304, which extends from a radially outer end of the annular part 302. The cylindrical portion 304, when in use, is housed inside the annular compartment 212. The test holders 301 are positioned along the cylindrical portion 304, so as to be able to move selectively and/or successively past the injector and analyzer. For example, the test supports 301 are part of a support 308, which comprises several chambers 310, separated from each other along a perimeter around the X axis. At least one test strip is received in a chamber 310.

The chambers 310 are arranged side by side in the shape of a right circular cylinder of at least 80% of the circle. To allow light to pass through, the rotatable holder 308 comprises at least one opening 312 per chamber 310 (shown in the upper left zoom where the rotatable holder is represented as transparent). The chambers 310 are all equidistant from the axis of rotation X, so that the injector can selectively inject urine once the desired chamber is positioned at the desired location facing the injector. The injector can move towards chamber 310, for example with the aid of a motor (referenced 704 on FIG. 7) and pierce a cover closing chamber 310 (visible on FIG. 4). A drain opening 314 is provided in the rotatable holder 300 to enable urine from the injector to be drained into the drain circuit, and thus to the outside of the device 100, via the drain port 219 located on the case 204.

The annular part 302 of the rotatable holder 300 remains outside the annular compartment 212 to reinforce the cylindrical part and/or rotate the cartridge 202. To this end, the annular part 302 may comprise a mechanical coupling 306, which cooperates with a shaft of the station 200.

Dimensions relating to the cartridge 202 are disclosed in the aforementioned documents. The maximum dimension of the device 100 transverse to the axis of rotation X is less than 15 cm, or even less than 10 cm. The maximum dimension of the device along the axis of rotation X is less than 5 cm.

### Collection port

Collection port 218 is configured to receive urine flowing by gravity over the outer surface of case 204. Urine is collected directly on the front face 220 and rear face 222 of the case 204.

Collection port 218 is an opening configured to collect liquid, so that liquid can enter the urine analysis device. Collection port 218 is generally circular, with a diameter of for example between 0.3 mm and 2 mm. The diameter of the collection orifice can be chosen to maximize the volume of urine collected on the outer surface of the case 204.

As can be seen from the figures, the collection port 218 is located on the rear face 222. In this way, collection port 218 faces the inner wall 112 of the toilet when urine analysis device 100 is positioned in the toilet. In this position, the collection port 218 is hidden from view by the front face 220 of the case. The front face 220 visible to the user resembles a simple, uniform pebble, as already mentioned, with no singular points or holes. It should also be noted that this position prevents the introduction of contaminants or elements that could obstruct the fluidic circuit.

Collection port 218 is located on a lower part of rear face 222. By "on a lower part of the rear face", we mean "on the last quarter of the face along the Z direction from the lower end of the case 204". The “Z direction” refers to the vertical axis when the device is positioned in its intended use configuration within toilet bowl 106, with the “bottom” defined as the portion of the case oriented toward the base of the bowl and the “top” defined as the portion oriented toward the toilet seat 108. The “lower end” of the case therefore faces the bowl bottom during use and is opposite the uppermost edge 550 of the case. The lower end faces the bottom of the tray 106 when the case 204 is positioned in the tray. The lower end is opposite the top 550. This position corresponds to normal use. This position allows urine to be collected by gravity over most of the outer surface of case 204. Locating collection port 218 in this lower region enables urine flowing by gravity across the external surface of case 204 to converge and be captured efficiently. This geometry ensures that the port is positioned at a natural collection point for liquid accumulation during urination. In some embodiments, the port may be located within 20 mm of the bottom edge of the rear face 222, thereby maximizing gravitational collection. In alternative embodiments, the collection port may be placed exactly at the bottom edge itself to ensure immediate capture of downward-flowing liquid. This placement also reduces the risk of miscollection from incidental splashes at higher elevations and ensures that substantially all urine that has contacted the outer surface is directed toward the port. By locating the port on the rear face (facing the bowl wall 112), the opening is concealed from a user’s line of sight, preserving discretion, while still positioned for optimal fluid capture by gravity. For purposes of clarity, “collection port” as used here refers to any defined opening, orifice, or aperture on case 204 through which urine enters the internal reservoir or fluidic circuit, optionally covered by a filter or septum. The term excludes decorative recesses or non-functional features of the case.

FIG. 4 illustrates two variants of rear face 222 with collection port 218. According to a first variant shown in FIG. 4A) , which is described in detail in document WO2021175944, the rear face 222 is smooth with a particular geometry for collecting urine, in particular a recess in the lower part of the case. According to a second variant shown in FIG. 4B) , which is described in detail in document EP23192264 (filing number), the rear face 222 has a network of ribs which enable urine flows to be directed towards the collection port 218.

In particular, the distance separating the collection port 218 from a lower edge of the case 204 is less than 40 mm, for example less than 20 mm. As illustrated, according to a particular embodiment, the collection port 218 is arranged a few millimeters above the lower edge of the case 204. Alternatively, the collection port 218 may be located on the bottom edge (the bottom edge being defined when the device 100 is placed for use in a toilet).

Collection port 218 may be covered by a mesh filter. The mesh filter is, for example, oblong in shape and covers the collection port 218. The average mesh size of the filter is, for example, 20 microns. The mesh filter prevents the introduction of contaminants or elements likely to obstruct the fluidic circuit and filters the urine received in the collection port 218. The filter mesh can be made of metal.

### Attachment

The case 204 can be held in position in the bowl by a fastener, of which FIG. 4B) shows part with a projecting stud 404 configured to cooperate with an arm that attaches to the rim of the bowl. Alternatively, a magnetic or other connection is possible.

### The analyzer

FIG. 5, which shows two cross-sectional views of two different embodiments A) and B) (the scale between the two embodiments is not maintained), illustrates in particular the interaction between the cartridge 202 and the station 200 at the level of the analyzer 500, with two embodiments of the analyzer. The fluidic architecture is also different between embodiments A) and B), but this is independent of the analyzer.

The analyzer 500 shown in FIG. 5A) comprises a light source 502 (e.g. two light sources) and at least one optical sensor 504, here in the form of a CCD (Charge Couple Device). Light travels from the light source 502 to the optical sensor 504, passing through the cartridge 202 and in particular the cylindrical part 304, the opening 312 of the holder 308, the test holder 301 and thus the urine reagent on the test holder 301.

In an embodiment, the analyzer 500 is configured to measure the absorbance of a portion of the test supports 301 (in particular the test line and/or the control line of a strip as will be explained later). Absorbance is detected by the light source (e.g. an LED), which can pass light through the strip, and the optical sensor, which receives the spectrum at around ten wavelengths.

The analyzer 500 of FIG. 5B) comprises an optical sensor in the form of a camera 506 capable of detecting a change in color, in particular a change in color intensity of the reagent, and therefore here of part of the test media 301 (in particular the test line and/or the control line of a strip). The camera can detect color in RGB values, for example. A light source can be provided so that the camera 406 can better identify colors.

FIG. 5B) illustrates in particular the aforementioned reservoir referenced 524. The reservoir 524 is formed here by the case 204: the collection port 218 is elongated to create a storage volume, which is the reservoir. The collection port 218, at the end of the reservoir 522 on the inside of the case, is closed by a septum 522. A grid (not visible) can be positioned at the reservoir inlet to filter debris.

The reservoir is outside case 204, so that urine flowing over the case can be collected freely. The reservoir therefore communicates with the outside of the station, and selectively with the inside (via the collection port).

### The injection end

FIG. 5 also illustrates an injection end 510 in FIG. 5A) and an injection end 520 in FIG. 5B). Different numerical references are used because the associated fluidic circuits may be different. This will be described in detail later.

The injection end 510, 520 may comprise a needle, in particular a beveled one, to be able, for example, to pierce the sealed chambers and/or pass through the septum.

The injection end 510, 520 can be moved between several positions. A distinction is made between a neutral position, in which the injection end does not cooperate with the reservoir, the drain circuit or the analysis region (and therefore does not pass through the space 212), an injection position, in which the injection end penetrates the space 212 to inject urine onto an analysis region 508, and a third position, the function of which depends on the fluidic circuit and which will be described later: the third position can be a sampling position, a drain position or a sacrifice position (this position being identical to the drain position).

In the neutral position, the injection end 510, 520 is, in the embodiment illustrated in the figures, located radially inside space 212. This maximizes the radius of the annular compartment while minimizing the size of station 200.

### Fluidic circuit

The urine analysis station comprises a fluidic circuit for conveying urine running down the case 204 to the analysis region 508, several embodiments or variants of which are shown in FIGS. 6 to 15. In particular, two particular embodiments are illustrated in FIGS. 6 and 7 (the cartridges 202 are not shown for simplicity of the figures) and other embodiments are illustrated in FIGS. 8 to 15. Different numerical references are used because the fluidic circuits are different.

The fluidic circuits shown all comprise a reservoir, a sampling end, which collects urine from the reservoir via the collection port 218, of the piping, an injection end 510, 520, and a drain end, which comprises the drain port 219. The drain end typically comprises piping connected to the drain port. The drain end serves to divert fluid in the fluidic circuit, in particular so that it is not injected into the analysis region. The drained fluid has no particular function. Because of the different fluidic architectures, so-called "drained" fluid may not yet have passed the drain port 219 (but may do so later, once injection has been completed or a cleaning sequence has been initiated).

FIGS. 6 and 7 illustrate respectively schematically and more realistically a fluidic circuit 600 which is structurally similar to that of documents WO2023036805, WO2023036806, WO2023036808, WO2023036809.

FIGS. 8 to 15 illustrate schematically and more realistically different fluidic circuit modes and variants.

### Fluid presence sensor

As shown as an example in FIG. 12 (but not visible in FIG. 7), station 200 may comprise at least one fluid presence sensor 1202, 1204 along the fluidic circuit. In an embodiment, station 200 comprises two fluid presence sensors 1202, 1204 spaced along the fluidic circuit and thus defining a reference section Sref (whose predetermined volume is known by control circuitry 1600), which ensures that a minimum volume of urine (the predetermined volume) has been collected. For example, the predetermined volume is between 40 and 50 microliters (particularly around 40 microliters). By measuring the fluid travel time between the two fluid presence sensors 1202, 1204, control circuitry 1600 can calculate the pump flow rate.

The one or two fluid presence sensors 1202, 1204 may comprise electrodes or optical probes. Document WO2022184984 describes such sensors (in particular the optical sensor) in detail.

Any fluidic circuit described herein may comprise at least two fluid presence sensors 1202, 1204.

Fluidic circuits, embodiments and variants

Several other fluidic circuit embodiments and variants will now be described.

### Definitions

The term "collection" means the introduction of urine into the fluidic circuit via the sampling end, from the reservoir, in particular via the collection port. The total volume of urine collected per micturition may be between 100 and 500 microliters, for example around 200 microliters.

The term "injection" refers to the introduction of urine into the analysis region via the injection tip, i.e., in particular, bringing urine into contact with a reagent (injecting step). Injection takes place during an injecting step, when the injection tip is in the injection position.

The term "drain" means to set aside in the hydraulic circuit, so that liquid can no longer be injected into the analysis region (drain step). Draining can include urine, which remains in the fluidic circuit temporarily, before being drained off later. Draining takes place during a drain step.

The term "first urine volumes collected" refers to the first urine volumes that enter the fluidic circuit through the collection port 218. Typically, the first urine volumes collected are between 50 and 150 microliters.

The term "last urine volumes collected" refers to the last urine volumes to enter the fluidic circuit through the collection port 218. Typically, the last urine volumes collected are between 50 and 150 microliters.

The expression "intermediate volumes of urine collected" refers to the intermediate volumes of urine that enter the fluidic circuit through the collection port 218, i.e. neither the first volumes collected nor the last volumes collected". Typically, intermediate urine volumes are between 50 and 150 microliters.

The term "injected urine volumes" refers to urine volumes that are brought into the reaction zone by the injection tip, i.e. in contact with urine reagent. The volume of urine injected (once or several times over the same analysis region) can be between 10 and 50 microliters.

In FIGS. 6 and 8 to 10, hollow arrows symbolize the presence of urine and the direction of fluid flow, while solid arrows symbolize movement of the injection tip or cartridge.

Thanks to fluid presence sensors 1202, 1204, control circuitry 1600 can measure the value of the pump flow rate. In addition, control circuitry 1600 knows the various internal volumes of the fluidic circuit (distance and/or cross-section of fluidic circuit components), which are predetermined. For example, the volume between the fluid presence sensor 1202 closest to the sampling end and the sampling end is between 50 and 80 microliters (e.g. around 70 microliters). Thus, control circuitry 1600 knows the flow rate of urine in the fluidic circuit, as well as the position of urine in the fluidic circuit relative to the fluid presence sensors (at the first sensor, between the two sensors, at the second sensor, after the second sensor; relative terms).

The hydraulic circuits described here operate in microfluidics, where the forces of capillarity are greater than those of gravity. Consequently, in the absence of pump operation, the liquid remains stationary in the fluidic circuit. Furthermore, the volumes of urine collected do not mix spontaneously as they move through the fluidic circuit. Particle movement within the urine is considered to be negligible compared with the movement of the urine itself.

General description of embodiments

In a so-called "sacrificial" embodiment, the fluidic circuit is configured to sacrifice the first volumes of urine taken from the reservoir by the sampling end. In other words, the urine analysis device 100 does not inject into the analysis region the first volumes of urine collected, which are drained. The fluidic circuit of urine analysis device 100 is deliberately configured so that the first volumes of urine collected during a urination are not used for analysis but are instead diverted away from the analysis region and discarded. The term “first volumes of urine” refers to the initial portion of liquid entering the fluidic circuit immediately after the user begins urination, typically ranging from about 50 microliters to about 150 microliters depending on flow conditions. These initial volumes are more likely to contain contaminants (for example, residual urine from a prior use, debris in the collection port, or epithelial cells and microorganisms present in the urethral opening). In practice, the sacrificial configuration means that when urine is drawn in through the sampling end (the intake point of the fluidic circuit connected to the reservoir), the control system operates valves, pumps, or injector positioning such that the first-collected portion is directed to a drain path rather than to the analysis region. Only after this sacrificial discharge step is complete does the device permit later-collected volumes (sometimes referred to as “intermediate” or “last-collected” volumes) to reach the analysis region for testing. By ensuring that no urine from the initial sacrificial fraction is injected into the analysis region, the device achieves two technical effects: (i) it reduces the risk of cross-contamination between successive analyses, and (ii) it increases reliability of diagnostic results by ensuring that the test is performed on fresher, representative urine from the mid-stream portion of the urination. For clarity, the term “sacrificial” as used here does not imply waste of the overall urine sample, but specifically denotes the intentional exclusion of an initial fraction of the sample from analytical contact, with that fraction being expelled to a drain outlet or other non-analytical path.

In another, so-called "injector-sampler" embodiment, which may be complementary to the sacrificial embodiment of the fluidic circuit, the injection end is used as the sampling end, through which urine enters the fluidic circuit. In this configuration, a single port serves both to collect urine into the fluidic circuit and to later inject urine into an analysis region. More specifically, during a sampling step, urine enters the circuit directly through the injection end, which is positioned adjacent to or penetrating a urine reservoir or collection septum. During a subsequent injection step, the same component delivers the collected urine onto a test strip or into a chamber of the analysis region. When combined with the sacrificial embodiment, the injector-sampler configuration ensures that the injection end is inherently flushed with fresh urine during collection, while the sacrificial control logic further ensures that the first-collected portion is diverted to waste. Together, these design choices minimize cross-contamination and improve the reliability of test results. For clarity, as used herein: “injection end” refers to the distal portion of the fluidic circuit, typically a nozzle or needle, configured to expel urine into an analysis region, and “sampling end” refers to the intake point of the fluidic circuit where urine first enters from a reservoir or collection port.

In another, so-called "two-way", embodiment, which may be complementary to the sacrificial and injector-sampler embodiments, the fluidic circuit comprises a urine direction-changing or direction-reversal section, within which the collected urine changes direction of flow: a leading front of a volume of urine in the section for changing the direction of movement of the urine becomes a trailing front of the same volume of urine (and conversely, the trailing front becomes the leading front). In this configuration, the pump or equivalent flow-driving element is capable of operating in both forward and reverse directions. During the injection step, the pump reverses its operation so that the urine already contained in the circuit travels in the opposite direction to that in which it was sampled. This reversal causes an inversion of the order of the urine volumes within the circuit. Specifically, the “leading front” of a collected urine volume (i.e., the portion of urine that first entered the circuit during sampling or collected bolus of urine) becomes the “trailing end” during injection, while the portion that was last collected becomes the new leading front. In this way, the last-in, first-out (LIFO) principle is achieved: the cleaner, later-collected urine is injected first into the analysis region, and the initial volumes (which may contain contaminants) remain at the back of the circuit until they are later expelled to a drain. For clarity: “urine direction-changing section” refers to a defined portion of the fluidic circuit, typically a linear tubing segment between two valve or pump interfaces, in which flow direction is capable of being reversed under pump control, and “leading front” refers to the foremost meniscus or boundary of urine in the circuit during forward flow, while “trailing front” refers to the rearmost boundary of the same volume. This two-way embodiment may be used independently, or in combination with the sacrificial and injector-sampler embodiments. When combined, the circuit ensures that the injection end is rinsed, that initial volumes are not injected, and that the most recently collected urine reaches the analysis region first, further improving analytical accuracy and reducing cross-contamination.

Various examples of fluidic circuits verifying at least one of the embodiments will be presented. FIGS. 8 to 11 are schematic and must be considered in the light of the more precise architecture provided in FIG. 6 or FIG. 7.

### Sacrificial embodiment

A fluidic circuit 600 conforming to the sacrificial embodiment (this embodiment is neither injector-sampler nor two-way) will now be described.

The fluidic circuit 600 shown in FIGS. 6 and 7 comprises, in series, the collection port 218, a sampling end 602, connected to piping 604 which leads to the pump 606, and then piping 608 connected to an injection end 510. Fluidic circuit 600 further comprises drain end 610, into which injection end 510 can discharge fluid to drain the circuit. The drain end 610 is thus opposite the sampling end 602, along the fluidic circuit 600.

In fluidic circuit 600, fluid flows in one and the same direction, from collection port 218 to injection end 510. Depending on the position of the injection end 510 (which is translationally movable), fluid in the fluidic circuit 600 can flow into the analysis region 508 (in injection position with a suitable position of the cartridge 202) or into the drain end 610 (in drain position). The pump 606 can therefore operate in a single direction of fluid flow, from the sampling end via the injection end to the drain end. This is referred to as a unidirectional direction of movement, from the tapping end 602 to the injection end 510.

A septum 512 may be provided to seal the collection port 218 from ambient moisture. The septum 512 can be passed through by the injection end 510 in the urine collection position.

Fluidic circuit 600 is linear, in the sense that there are no fluidic junctions or bifurcations, making it particularly simple, tight and easy to use.

The pump 606 and the position of the injection end 610 are controlled by control circuitry 1600.

In this fluidic circuit 600, the analysis region 508 and the drain end 610 are arranged in close proximity so that the injection end 510 can selectively discharge fluid into them. In particular, when the injection end 510 is movable in translation, the injection end 510, the analysis region 508 and the drain end 610 are aligned along the translation direction.

As illustrated in FIG. 6a) , during a sampling step, the pump 606 is activated and urine enters the sampling end 602 through the collection port 218, then via the piping 602, 606, to the injection end 510 (FIG. 6a) . The injection end may be in the standby or drain position.

The first volumes sampled off travel first through the fluidic circuit 600. As such, they may be contaminated by urine residues from a previous sampling. It is therefore preferable not to inject these first volumes of urine onto the urine reagent.

To this end, as illustrated in FIG. 6b), during a sacrifice step, control circuitry 1600 puts or holds the injection end 510 in the drain position, so that the injection end 510 communicates with the drain end 610. Control circuitry 1600 activates the pump to evacuate the first volumes of urine collected towards drain end 610.

Fluid presence sensors 1202, 1204, which provide information on the pump's flow rate, provide information on the evacuated volume (control circuitry 1600 can store the value of the fluidic circuit volume between a fluid presence sensor 1202, 1204 and the injection end).

Then, as shown in FIG. 6c), in a transitioning step, control circuitry 1600 moves the injection end 510 to a neutral position and positions the cartridge so that the desired analysis region 508 is in position to receive urine through the injection end 510.

Finally, as illustrated in FIG. 6d), during an injecting step, control circuitry 1600 reactivates pump 606 and injection tip 510 injects volumes of sampled urine, which correspond to intermediate volumes of sampled urine, into analysis region 508. In this way, the first and potentially contaminated volumes collected are sacrificed in the drain end and are not used for urine analysis.

Finally, as illustrated in FIG. 6e), during a drain step, control circuitry 1600 positions the injection end in the drain position to allow draining of fluidic circuit 1600. The injection end 510 thus discharges fluid into the drain end 610. In a preliminary transitioning step, the control circuitry 1600 can put the injection end 520 in a neutral position for the draining step. In this drain step, the first volumes taken are already in the drain end (or already drained via the drain orifice).

Fluidic circuit 600 is particularly simple to implement.

### Injector-sampler embodiment

Fluidic circuit 800 conforms to the injector-sampler embodiment.

In this mode, the injection end is used as the sampling end, i.e. urine flows through the sampling end in one direction for collection, then through the sampling end for injection, and the first volumes of urine collected remain on the first front. There is therefore no section for changing the direction of movement of the urine, within which the urine sampled changes direction of movement (with inversion of the front and rear faces, as described above).

Fluidic circuit 800 comprises in series the collection port 218, the sampling end 802 (which is the injection end 520) which piping 804 connects to a valve 806, which in turn is connected to piping 808 connected to a pump 810, which is connected to piping 812 connected to valve 806, connected to a drain end 814 connects to drain port 219.

Valve 806 is configured to reverse the connections between piping 804, 808 on one side and piping 812 and bleed end 814 on the other.

Valve 806 may be a so-called "4-way 2-position" valve, with 2 pairs of ports that are selectively connected to each other. Valve 806 may comprise a set of fluidically and functionally equivalent hydraulic elements.

Fluidic circuit 800 forms a loop between sampling end 802 and injection end 520.

In the fluidic circuit 800, the sampling end 802 is unitary with the injection end 520, in that fluid sampling from the collection port 218 is carried out by the injection end 520. Put another way, the injection end 520, configured to inject urine into the analysis region 508, serves as the sampling end 802.

In this fluidic circuit 800, the analysis region 508 and the reservoir 524 are arranged in close proximity so that the injection end 510 (which is also the sampling end 802) can selectively discharge urine into it or collect urine from it. In particular, when the injection end 520 is movable in translation, the injection end 520, the analysis region 508 and the reservoir 524 are aligned along the direction of translation.

As the fluidic circuit is at times disconnected from the collection port 218, the latter is sealed from the inside of the case by the septum 524 (visible in FIG. 5B), which the injection end 520 (here the sampling end 802) can pass through in the urine collection position. To this end, the injection end may comprise a needle, in particular a beveled needle.

Sensors 1202, 1204 for detecting the presence of urine are typically located at the level of pipe 808 (i.e. beyond valve 806).

As illustrated in FIG. 8a), during a sampling step, the control circuitry 1600 places the injection end 520 in the sampling position and the pump 808 sucks so that the urine enters the fluidic circuit 800. Piping 804, 808, 812 is long enough to store enough urine sampled (including the portion between the two fluid presence sensors 1202, 1204).

Then, as illustrated in FIG. 8b) , in a transitioning step, the control circuitry 1600 puts the injection end 520 in the injection position, once the analysis region 508 is arranged in front of the injection end 520. Control circuitry 1600 also drives valve 806 to reverse the positions and connect pipe 812 to pipe 804 via valve 806.

In FIG. 8c) , during an injecting step, control circuitry 1600 activates pump 810 again, and urine continues to flow in the same direction. As a result of the reversal of valve 806, the collected urine is redirected to the injection end 520, which then injects onto the analysis region 508.

Finally, as illustrated in position 8 d), during a draining step, control circuitry 1600 drives valve 806 to re-invert positions and thus enable draining of urine remaining in injection end 520 and piping 804, 808, 812 to drain port 219. Control circuitry 1600 can also put the injection end 510 in neutral position (notably to let air enter the circuit).

The hydraulic circuit 800 is compact, since the injection end 520 and the sampling end 802 are one and the same part. In addition, the injection end 520 is cleaned during sampling by the first sampled volumes.

In this variant, the first sampled volumes are the first injected volumes.

### Injector-sampler and sacrificial embodiment

FIG. 9 illustrates a variant 900 of the 800 fluidic circuit, which also conforms to the sacrificial embodiment. The numerical references are identical to those of fluidic circuit 800, but the logic for activating the valve and/or pump changes.

To further limit cross-contamination and avoid injecting the first sampled volumes, in a sacrificial step, control circuitry 1600 can activate the position of valve 806 of FIG. 9b) only once the first sampled urine volumes have passed valve 806 to reach the drain end 814, as represented by the arrow in the drain end of FIG. 9a) . In this way, the first volumes of urine collected are sacrificed and the volumes of urine injected are the intermediate volumes of urine collected.

During the injecting step (FIG. 9c) , the first urine volumes are therefore in the drain end 814 and can then be recirculated or conveyed into the piping 808, via the valve 806, to ultimatelybe drained, as described in the position of FIG. 9d). Thus, in the draining step (FIG. 9d ), the first volumes taken are already in the drain end (or already drained via the drain orifice).

### Two-way operation

Fluidic circuit 1000 of FIG. 10 conforms to the two-way embodiment, as well as, optionally, conforming to the sacrificial embodiment (notably in that it is structurally designed for natural implementation of the sacrificial embodiment).

In this embodiment, fluidic circuit 1000 comprises a urine direction changeover section, within which the collected urine changes its direction of flow, i.e. within the urine direction changeover section a leading front of a volume of urine becomes the trailing front of that volume of urine. The benefits of such a change of direction will be explained later.

The fluidic circuit 1000 comprises in series the collection port 218 connected to the sampling end 1002 which piping 1004 connects to a valve 1006, which is itself connected to piping 1008 connected to a pump 1010, which is connected to a drain end 1012. Furthermore, in parallel, valve 1006 is also connected to injection end 520 via piping 1014.

Valve 1006 is configured to selectively connect piping 1008 to piping 1004 (i.e. to the sampling end 1002) or piping 1014 (i.e. to the 520 injection end).

Valve 1006 can be a 2-way 2-position valve, with two inlets selectively connected to one outlet.

In this embodiment, the pump 1010 is configured to move the fluid in the fluidic circuit 1000 selectively in one direction or the other.

Fluid presence sensors 1202, 1204 are typically located in piping 1008 (i.e. beyond valve 1006).

As illustrated in FIG. 10a) , in a sampling step, the control circuitry 1600 sets (or holds) the valve 1006 in position connecting the sampling end 1002 to the piping 1008 and activates the pump 1010, so that the sampling end 1002 samples liquid from the reservoir 524. The sampled volumes enter piping 1008.

Once a sufficient quantity has been sampled, as illustrated in FIG. 10b) , in a transitioning step, control circuitry 1600 moves the injection end 520 into the injection position, once the analysis region 508 is positioned in front of the injection end 520.

Then, as shown in FIG. 10c) , in an injecting step, control circuitry 1600 reverses the direction of operation of pump 1010, so that urine changes direction within piping 1008, flowing back through valve 1006 to pipe 1014 and injection end 520.

The change of direction means that the first volumes of urine sampled, which were at the front of the urine front in the fluidic circuit, end up at the rear, and the last volumes of urine removed, which were at the rear of the urine front in the fluidic circuit, end up at the front.

In this way, the last or intermediate volumes of urine collected, which are less susceptible to cross-contamination, end up being the first volumes injected into the analysis region 508. In the first case, this is LIFO (last in - first out) logic.

In FIG. 10d) , in a draining step, control circuitry 1600 again changes the direction of operation of pump 1010, so that urine still present in fluidic circuit 1000 is drained towards drain port 219. Control circuitry 1600 can also reposition valve 1006 to drain the entire circuit. In a preliminary transitioning step, control circuitry 1600 can set the injection end 520 to the neutral position for the draining step.

In this draining step, the first sampled volumes pass through the draining end.

In one variant, the position shown in FIG. 10c) is implemented while urine is still being collected in the sampling end 1002 and/or the piping 1004 (these are the last volumes collected). In this case, the volumes of urine injected correspond to intermediate volumes of urine collected. This urine is of a similar quality to the last urine volumes collected.

Fluidic circuit 1000 thus comprises a section for changing the direction of movement of urine 1014, within which the fronts of a volume of urine are reversed: the front becomes the rear and vice versa. The urine direction change section is here a portion of piping 1008.

Fluidic circuit 1000 can thus present a linear (i.e. seamless) section for changing the direction of movement of the urine, within which the fluid reverses its direction of circulation or conveyance: the fluid therefore flows in the other direction (this is to be distinguished from a situation where a loop allows the fluid to flow in the opposite direction, but the fluid does not change direction in the portion, the loop not being a linear portion since a junction is required - see, for example, pipe 804 in FIG. 8, through which urine flows in both directions, but the urine loops through valve 806 and pipe 808, 812). The direction of fluid flow is taken to mean the direction in which the flow rate is positive, i.e. the direction in which the majority of the elements making up the fluid flow.

In the embodiment shown in FIG. 10, the sampling end 1002 and the injection end 520 are two separate parts.

When two fluid presence sensors 1202, 1204 are arranged along the fluidic circuit, the urine displacement changeover section may comprise the portion of the fluidic circuit between the two fluid presence sensors 1202, 1204. In fact, the reversal of direction can be performed once both fluid presence sensors 1202, 1204 have identified urine, so that the control circuitry knows that a sufficient volume of urine has been collected.

Two-way and sacrificial embodiments

The fluidic circuit 1000 shown in FIG. 10 can also be designed as a sacrificial circuit.

In this respect, it is sufficient for the injecting step in FIG. 10c) to stop before the first volumes of collected urine are injected into the analysis region 508. Draining then takes place as shown in FIG. 10c) .

Two-way, typically sacrificial, injector-sampler embodiment

A fluidic circuit 1100 conforming to the injector-sampler, two-way and, optionally, sacrificial modes of implementation (in particular in that it is structurally designed for natural implementation of the sacrificial embodiment) will be described.

This fluidic circuit 1100 offers numerous benefits: cross-contamination is minimized as injected urine volumes only pass through portions of the fluidic circuit that have been rinsed by the first sampled volumes.

FIG. 11 schematically illustrates fluidic circuit 1100; FIG. 12 illustrates an implementation version of fluidic circuit 1100 in device 100 (with relative positioning of components) and FIGS. 13 to 15 represent a simplified version of FIG. 12 with the steps of FIG. 11.

The fluidic circuit 1100 comprises in series the collection port 218 (with here the septum 522 and the reservoir 524 described in relation to FIG. 8), connected to the sampling end 1102 which is also the injection end 520, itself connected to piping 1104 which leads to the pump 1106, then a drain end 1108 which connects to the drain port 219.

Pump 1106 is configured to move fluid in fluidic circuit 1100 selectively in one direction or the other.

The fluidic circuit 1100 is linear, in the sense that there are no fluidic junctions or bifurcations, making it particularly simple, tight and easy to use.

In this fluidic circuit 1100, the analysis region 508 and the reservoir 524 are arranged in close proximity so that the injection end 520 (which is also the sampling end 802) can selectively discharge urine into it or collect urine from it. In particular, when the injection end 520 is translationally movable, the injection end 520, the analysis region 508 and the reservoir 524 are aligned along the translation direction.

The two fluid presence sensors 1202, 1204 are typically arranged at pipe 1104 or 1108.

As illustrated in FIGS. 11a) and 13, in a sampling step, control circuitry 1600 sets or holds injection end 520 in the sampling position and activates pump 1106 so that urine is sampled from collection port 218 to injection end 520 and then to pipe 1104. Volumes of urine are thus sampled into tubing 1104. Typically, sampling takes place until the fluid presence sensor 1202, 1204 detects urine. This ensures that the piping volume between the two urine sensors is filled with urine.

Then, as illustrated in FIG. 11b), in a transitioning step, the control circuitry 1600 moves the injection end 520 into the injection position, once the analysis region 508 is arranged in front of the injection end 520.

Then, as illustrated in FIGS. 11c) and 14, in an injecting step, the control circuitry 1600 activates the pump 1106 so that the urine taken changes direction of circulation or conveyance within the pipe 1104. This results in a section 1110 for changing the direction of movement of the urine, which comprises at least a portion of pipework 1104 (and which may even comprise the injection end 520). In this case, section 1110 comprises in particular the portion of the fluidic circuit (here of pipework 1104) comprised between the two fluid presence sensors. The injection end 520 is therefore flowed through by urine in one direction, during sampling (the first volumes sampled being on the front face) and in the other direction, during injection (the last volumes of urine sampled being on the front face).

Injection is typically controlled so that not all the urine collected is injected into the analysis region 508. In particular, control circuitry 1600 stops pump 606 before the first sampled volumes are injected. In this case, the fluidic circuit conforms to the sacrificial embodiment.

Finally, as illustrated in FIGS. 11d) and 15 , in a draining step, control circuitry 1600 changes the direction of operation of pump 606 to drain fluidic circuit 1100 by directing remaining urine to drain port 219. In a preliminary transitioning step, control circuitry 1600 can set the injection end 520 to the neutral position for the draining step.

Thanks to this change of direction, the last urine volumes collected or the intermediate volumes collected (which are the cleanest because the first urine volumes collected have cleaned the fluidic circuit 1100) become the injected urine volumes. Moreover, thanks to the fact that the injection end 520 is the sampling end 1102 and thanks to the two-way fluidic circuit 1100, the path travelled in fluidic circuit 1100 by the injected urine volumes has been cleaned by the first urine volumes sampled.

Finally, by stopping the injection before the first sampled urine volumes are injected, it is ensured that the first sampled urine volumes are sacrificed.

This fluidic circuit 1100 offers a number of benefits, which will be discussed in greater detail later. In particular, this fluidic circuit 1100 significantly reduces the risk of cross-contamination for three reasons: the fluidic circuit used for injection has been cleaned during sampling (since the injection end serves as the sampling end). Finally, this fluidic circuit 1100 allows the injection of recently collected urine into the analysis region 508, i.e. urine which is flowing through a fluidic circuit which has already been completely cleaned.

The first volumes of urine injected correspond to the last volumes of urine collected or to the intermediate volumes of urine collected, and the portion of the fluidic circuit traversed by the injected volumes has been entirely traversed by the first volumes of urine collected (notably the injection end 520). The details of this circuit will be explained later, including variants.

In the embodiments described, the analysis region 508 can be brought into position by rotating the cartridge 202 in space 212.

### Preload

For the above-described embodiments, with the exception of that shown in FIGS. 6 and 7, a variant with pre-charging can be implemented. During sampling, a mixture of air and urine may be sampled, due in particular to the irregularity of urine flow into the reservoir. As a result, the fluidic circuit may alternate between urine and air. For injection, it is desirable to expel only urine.

Thanks to the two fluid presence sensors 1202, 1204, the sampling stage can be stopped when the reference section Sref between the two fluid presence sensors 1202, 1204 is filled with urine (without the presence of an air front). Pre-charging consists in draining urine through the sampling end 510, 520, so that the urine present in the reference section Sref is moved to the sampling end. In this way, the pre-load can include an evacuation of 80 microliters of urine. This volume is determined as a function of the volumes mentioned in the description. The volumes injected are therefore intermediate volumes of urine sampled.

In the embodiment shown in FIGS. 8 or 9 , the pre-charging step takes place before the transitioning step of FIGS. 8b) or 9b). In this case, the control circuitry 1600 leaves the injection end 520 in the sampling position, but switches the valve 806 to reverse the positions relative to the positions during the sampling step. The pump 810 is then reactivated to empty a volume of urine as described above. In this way, the first volumes collected (for the embodiment of FIG. 8) or intermediate volumes of urine collected (for the embodiment of FIG. 9) are discharged into the reservoir 524. Then, the transitioning step of FIG. 8b) is implemented (except that valve 806 is already in place).

In the embodiment shown in FIG. 10, wherein a bleed end such as that shown in FIGS. 8, 9 and 11 is additionally provided, the pre-charging step takes place before the transitioning step of FIG. 10b) . In this case, the control circuitry 1600 puts the injection end 520 in a drain position (not visible in FIG. 10, the drain end is not directly accessible through the injection end) and switches the valve 1006 to reverse the positions with respect to the positions during the sampling step. Pump 1010 is then activated in reverse to empty a volume of urine as described above into the drain end. In this way, the last volumes sampled (for the embodiment shown in FIG. 9) or intermediate volumes of urine sampled (for the variant not shown where urine is present in piping 1004) are discharged into reservoir 524. Then, the transitioning step of FIG. 10b) is implemented (except that valve 1006 is already in place).

In the embodiment shown in FIG. 11, the pre-charge step is performed before the transitioning step of FIG. 11b) . In this case, control circuitry 1600 leaves the injection end 520 in the sampling position. Pump 1106 is then activated in the opposite direction to empty a volume of urine as described above into reservoir 524. In this way, the last volumes collected are discharged into reservoir 524. Then, the transitioning step of FIG. 10b) is implemented (except that valve 1006 is already in place).

Architecture and control circuitry

FIG. 16 schematically illustrates a station 200 with control circuitry 1600. Control circuitry 1600 comprises a processor 1602, a memory 1604 (RAM or ROM, e.g. non-volatile) and an I/O ("in/out") interface 1606 for exchanging data. The term “control circuitry” as used herein refers to one or more microcontrollers, processors, or equivalent computing devices, together with associated memory and interfaces, adapted to execute stored instructions and manage the operation of the station.

Memory 1604 can store programs executable by processor 1602. When executed by the processor 1602, these programs control actuation of pumps, motors, and sensors, as well as data acquisition from the analyzer.

Station 200 further comprises a battery 1608 configured to supply power to electrical or electronic components of station 200, including fluidic control elements and wireless communication circuits.

In particular, control circuitry 1600 can control pump (referenced 606, 810, 1010, 1106), motor 702 for moving cartridge 202 in station 200, motor 704 for moving injection end 510, 520. In particular, control circuitry 1600 can exchange data with fluid presence sensor(s) 1202, 1204 positioned along the fluidic circuit to detect the presence or absence of urine, thereby informing control logic regarding when to initiate sampling, injection, or drainage.

Station 200 may also include a wireless communication module 1612 (e.g. a BlueTooth or BlueTooth Low Energy module), connected to control circuitry 1600. Module 1612 enables data to be exchanged (transmitted and received), via a communication network 1614 with a mobile terminal 1616 (e.g. a smartphone) and/or a remote server 1618. The communication network 1614 may be wireless and/or wired and/or a mixture of both. Urine data can thus be sent to the server 1618 and then transmitted to the mobile terminal 1616 (or communicated directly to the mobile terminal 1616, which then transmits it to the server). Conversely, station 200 can receive updates from remote server 1618 or mobile terminal 1616, configuration data, or user instructions from the mobile terminal 1616 or server 1618, thereby supporting remote monitoring and adaptive control of the device.

### Generalization

The fluidic circuit described in the present description applies in the same way in a device whose cartridge does not move in rotation but, for example, in translation.

The injection end, described as translational, may be movable in another way, for example in rotation.

As described, urine analysis can be performed via a reagent or on the urine directly.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles "a" and "an" may be employed in connection with various elements and components, processes or structures described herein. This is merely for convenience and to give a general sense of the compositions, processes or structures. Such a description includes "one or at least one" of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A urine analysis station comprising:
a case configured to be disposed entirely within a bowl of a toilet and to receive a stream of urine, the case being configured to receive therein at least one analysis region adapted to receive urine for analysis,
a urine reservoir, configured to receive urine from a user's urination,
a fluidic circuit inside the case for conveying urine in the station from the urine reservoir to an analysis region, wherein the fluidic circuit comprises an injection end configured to inject urine into an analysis region and a sampling end configured to sample urine from the reservoir into the fluidic circuit,
an analyzer, mounted inside the case, and configured to obtain data relating to the urine in the analysis region,
wherein the injection end is used as the sampling end.

2. The station according to claim 1, wherein the reservoir and the at least one analysis region are arranged in close proximity in the station.

3. The station according to claim 1, wherein the reservoir and the at least one analysis region are arranged in alignment with a translation axis of the injection end.

4. The station according to claim 1, wherein the injection end is movable between notably an injection position, during which the injection end injects urine into the analysis region, and a sampling position, during which the injection end samples urine from the reservoir.

5. The station according to claim 4, wherein the injection end is a syringe.

6. The station according to claim 1, comprising a pump configured to convey urine in the fluidic circuit in one direction during a step of sampling urine from the reservoir by the sampling end and in the opposite direction during a step of injecting urine into the analysis region by the injection end.

7. The station according to claim 1, wherein the fluidic circuit is linear.

8. The station according to claim 1, configured to sacrifice the first volumes of urine taken from the reservoir by the sampling end, so that the urine station does not inject the first volumes of urine taken into the analysis region.

9. The station according to claim 1, wherein the fluidic circuit comprises a section for changing the direction of movement of the urine,
within which urine changes direction of flow, within which a leading front of a volume of urine becomes the trailing front of that volume of urine.

10. The station according to claim 1, wherein the logic of the fluidic circuit is last in - first out (LIFO) so that the last volumes of urine drawn by the sampling end from the reservoir are the first volumes of urine injected into the analysis region by the injection end.

11. The station according to claim 1, comprising a space positioned inside the case, configured to at least partially receive a cartridge comprising the at least one analysis region.

12. The station according to claim 1, wherein the reservoir is accessible from the sampling end via a septum configured to be traversed by the sampling end.

13. The station according to claim 1, wherein a same port of the fluidic circuit is provided to both collect urine from the reservoir and inject urine into the at least one analysis region.

14. The station according to claim 1, wherein the fluidic circuit is configured to divert a first-collected portion of urine away from the at least one analysis region to a drain path such that the first-collected portion is not injected into the at least one analysis region.

15. The station according to claim 1, further comprising a pump configured to operate in forward and reverse directions to reverse a flow direction of urine within the fluidic circuit so that a last-collected portion of urine is delivered to the at least one analysis region before a first-collected portion.

16. The station according to claim 1, further comprising first and second fluid presence sensors arranged along the fluidic circuit to define a reference section of known volume, the station being configured to commence injection after the reference section is detected as containing urine.

17. The station according to claim 1, wherein the urine reservoir is accessible through a septum and the injection end is movable along a translation axis between a sampling position at the septum and an injection position aligned with the at least one analysis region.

18. A device comprising a station according to claim 1, and a removable cartridge of the station, wherein the at least one analysis region is mounted on the cartridge.

19. A method of urine analysis using a station according to claim 1, comprising:
sampling urine from the reservoir through the sampling end, and
injecting urine into the analysis region through the injection end.

20. The method according to claim 19, comprising, in use, between the sampling and the injecting, a pre-charging step, wherein sampled urine is returned to the reservoir through the sampling end.
